SEQUENCE LISTING (4 sequences)

SEQ ID NO: 1 - DNA, 1773 bp, Bacillus licheniformis ATCC 68614
  CDS (233)..(1372)
  mat_peptide (566)..(1372)
ggatcctcgggacctctttccctgccaggctgaagcggtctattcatactttcgaactgaacatttttctaaaacagttattaataaccaaaaaattttaaattggtcctccaaaaaaataggcctaccatataattcattttttttctataataaattaacagaataattggaatagattatattatccttctatttaaattattctgaataaagaggaggagagtgagtaatgatgaggaaaaagagtttttggcttgggatgctgacggccttcatgctcgtgttcacgatggcatcgatcgcatcggctgctgaggaagcaaaagaaaaatatttaattggctttaatgagcaggaagctgtcagtgagtttgtagaacaagtagaggcaaatgacgaggtcgccattctctctgaggaagaggaagtcgaaattgaactgcttcatgagtttgaaacgattcctgttttatccgttgagttaagcccagaagatgtggacgcgcttgaacttgatccagcgatttcttatattgaagaggatgcagaagtaacgacaatggcgcaaacaatcccatggggaattagccgtgtgcaagccccggctgcccataaccgtggattgacaggttctggtgtaaaagttgctgtcctcgatacaggtatttccactcatccagacttaaatattcgtggtggcgctagctttgtaccaggggaaccatccactcaagatgggaatgggcatggcacgcatgtggccgggacgattgctgctttaaacaattcgattggcgttcttggcgtagcgcctagtgcggaactatacgctgttaaagttttaggagccgacggtagaggtgcaatcagctcgattgcccaagggttggaatgggcagggaacaatggcatgcacgttgctaatttgagtttaggaagcccttcgccaagtgccacacttgagcaagctgttaatagcgcgacttctagaggcgttcttgttgtagcggcatctgggaattcaggtgcaagctcaatcagctatccggcccgttatgcgaacgcaatggcagtcggagctactgaccaaaacaacaaccgcgccagcttttcacagtatggcccagggcttgacattatggcaccaggggtaaacattcagagcacatacccaggttcaacgtatgccagcttaaacggtacatcgatggctactcctcatgttgcaggtgcagcagcccttgttaaacaaaagaacccatcttggtccaatgtacaaatccgcaaccatctaaagaatacggcaacgagcttaggaagcacgaacttgtatggaagcggacttgtcaatgcagaagcggcaacacgctaatcaataaaaaaaccgtgtgcgcttaaagggcacagctttttttgtgtatgaatcgaaaaaagagaacagatcgcaggtctcaaaaatcgagcgtaaagggttgtttaaagctctttacgctcgcaggtcttatcgctatacaatggaaaattcacgtcttttgactttcatggcatatttatttaagtattcgtttgctttttcgtactctccgtttttctggtacctccttctactatgggaaaggtctgatcaatgtcgaagctgccgctcaataacatattctaacaaatagcatatagaaaaagctagtgtttttagcactagctttttcttcattctgatgaaggttgttcaatattttgaatccgttccatgatcgtcgggtaccgagctct

SEQ ID NO: 2 - protein, 380 aa, Bacillus licheniformis ATCC 68614
MMRKKSFWLGMLTAFMLVFTMASIASAAEEAKEKYLIGFNEQEAVSEFVEQVEANDEVAILSEEEEVEIELLHEFETIPVLSVELSPEDVDALELDPAISYIEEDAEVTTMAQTIPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGPGLDIMAPGVNIQSTYPGSTYASLNGTSMATPHVAGAAALVKQKNPSWSNVQIRNHLKNTATSLGSTNLYGSGLVNAEAATR

SEQ ID NO: 3 - DNA, 1143 bp, Artificial Sequence
  Bacillus lentus alkaline protease S3T/V4I/V199I/L211G
atgatgaggaaaaagagtttttggcttgggatgctgacggccttcatgctcgtgttcacgatggcatcgatcgcatcggctgctgaggaagcaaaagaaaaatatttaattggctttaatgagcaggaagctgtcagtgagtttgtagaacaagtagaggcaaatgacgaggtcgccattctctctgaggaagaggaagtcgaaattgaactgcttcatgagtttgaaacgattcctgttttatccgttgagttaagcccagaagatgtggacgcgcttgaacttgatccagcgatttcttatattgaagaggatgcagaagtaacgacaatggcgcaaacaatcccatggggaattagccgtgtgcaagccccggctgcccataaccgtggattgacaggttctggtgtaaaagttgctgtcctcgatacaggtatttccactcatccagacttaaatattcgtggtggcgctagctttgtaccaggggaaccatccactcaagatgggaatgggcatggcacgcatgtggccgggacgattgctgctttaaacaattcgattggcgttcttggcgtagcgcctagtgcggaactatacgctgttaaagttttaggagccgacggtagaggtgcaatcagctcgattgcccaagggttggaatgggcagggaacaatggcatgcacgttgctaatttgagtttaggaagcccttcgccaagtgccacacttgagcaagctgttaatagcgcgacttctagaggcgttcttgttgtagcggcatctgggaattcaggtgcaagctcaatcagctatccggcccgttatgcgaacgcaatggcagtcggagctactgaccaaaacaacaaccgcgccagcttttcacagtatggcgccgggcttgacattgtggcacccggggtaaacattcagagcacatacccaggttcaacgtatgctagcggaaacggtacatcgatggctactcctcatgttgcaggtgcagcagcccttgttaaacaaaagaacccatcttggtccaatgtacaaatccgcaaccatctaaagaatacggcaacgagcttaggaagcacgaacttgtatggaagcggacttgtcaatgcagaagcggcaacacgctaa

SEQ ID NO: 4 - protein, 380 aa, Artificial Sequence
  Bacillus lentus alkaline protease S3T/V4I/V199I/L211G
MMRKKSFWLGMLTAFMLVFTMASIASAAEEAKEKYLIGFNEQEAVSEFVEQVEANDEVAILSEEEEVEIELLHEFETIPVLSVELSPEDVDALELDPAISYIEEDAEVTTMAQTIPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNIQSTYPGSTYASGNGTSMATPHVAGAAALVKQKNPSWSNVQIRNHLKNTATSLGSTNLYGSGLVNAEAATR

(19) United States
(12) Patent Application Publication
(10) Pub. No.: US 2005/0026269 A1
(43) Pub. Date: Feb. 3, 2005

(54) Novel alkaline protease variants and detergents and cleaning agents containing said novel alkaline protease variants

(76) Inventors: Beatrix Kottwitz, Dusseldorf (DE); Karl-Heinz Maurer, Erkrath (DE); Roland Breves, Mettmann (DE)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/476,463

(22) PCT Filed: Apr. 24, 2002
(86) PCT No.: PCT/EP02/04489

(30) Foreign Application Priority Data
May 2, 2001 (DE) 101214634

(51) Int. Cl. C07H 21/04; C12N 9/56; C11D 3/386; C12N 15/74

(52) U.S. Cl. 435/222; 435/69.1; 435/252.31; 435/320.1; 510/320; 536/23.2

(57) ABSTRACT

Described herein are novel alkaline protease variants derived from subtilisin. These variants have, with respect to the amino acid sequence of Bacillus lentus subtilisin, variations at amino acid positions 199 and 211, and at least one modification that contributes to the stabilization of the molecule, the modification preferably being variations at amino acid positions 3 and/or 4. Preferably, the variant is B. lentus alkaline protease S3T/NV4I/V199I/L211G. Also described are detergents and cleaning agents comprising the novel alkaline protease variants. Methods of use employing the novel alkaline protease variants are also described.

The present invention relates to novel alkaline protease variants which are derived from natural or modified subtilisin proteases. According to the numbering of the subtilisin from Bacillus lentus, said variants have, compared to the previously known subtilisins, the two amino acid positions 199I and 211G and at least one modification, preferably, after point mutation, the amino acids threonine in position 3 and/or isoleucine in position 4, which modification contributes to the stabilization of the molecule. Particular preference is given to the variant B. lentus alkaline protease S3T/V4I/V199I/L211G. These variants distinguish themselves from other protease variants by an improved contribution to the cleaning performance of detergents and cleaning agents. Therefore, in addition to said enzymes, the present invention relates to their use in various technical processes and, in particular, to detergents and cleaning agents containing said novel alkaline protease variants.

Proteases of the subtilisin type (subtilases, subtilopeptidases, EC 3.4.21.62) are classed as belonging to the serine proteases, due to the catalytically active amino acids. They are naturally produced and secreted by microorganisms, in particular by Bacillus species. They act as unspecific endopeptidases, i.e. they hydrolyze any acid amide bonds located inside peptides or proteins. Their pH optimum is usually within the distinctly alkaline range. A review of this family is provided, for example, in the paper “Subtilases: Subtilisin-like Proteases” by R. Siezen, pages 75-95 in “Subtilisin enzymes”, edited by R. Bott and C. Betzel, New York, 1996. Subtilisins are suitable for a multiplicity of possible technical uses, in particular as active ingredients of detergents or cleaning agents.

Apart from enzymes such as, for example, amylases, lipases or cellulases, proteases have already been used for decades as active components in detergents and cleaning agents. They have the ability to break down proteinaceous soilings on the material to be cleaned such as, for example, textiles or dishes. Owing to their relatively high solubility, the hydrolysis products are washed away with the wash liquor or are attacked, dissolved, emulsified or suspended by the other components of the detergents or cleaning agents. Thus, synergistic effects between the enzymes and the other components of the detergents and cleaning agents in question can arise.

Owing to their favorable enzymic properties such as stability or pH optimum, subtilisins stand out among the detergent and cleaning agent proteases. The most important subtilisin proteases currently used in detergents, which are partly natural molecules, partly variants derived from these wild-type enzymes by mutagenesis, are listed below.

Subtilisin BPN′ which originates from Bacillus amyloliquefaciens and B. subtilis, respectively, has been disclosed in the studies by Vasantha et al. (1984) in J. Bacteriol. Volume 159, pp. 811-819 and by J. A. Wells et al. (1983) in Nucleic Acids Research, Volume 11, pp. 7911-7925. The patent applications WO 95/07991 and WO 95/30010 present, for example, variants with reduced binding to the substrate at a simultaneously increased rate of hydrolysis, which were obtained as a result of point mutations in the loop regions of said enzyme. The patent application WO 95/29979, for example, discloses detergents containing such BPN′ variants.

Two of the amino acid positions considered in the present patent application, namely positions 3 and 4, are not located in loop regions; the residues 199 and 211 are homologous to positions 205 and 217, respectively, of BPN′, which are located in loop 6 of the molecule, as is described in the applications mentioned, for example. Said loop is involved in substrate binding. Position 205 in BPN′ contains an isoleucine (I) by nature. Application WO 95/07991 proposes numerous possible amino acids with which the tyrosine (Y) located by nature in position 217 can be replaced, but not in conjunction with stabilizing mutations of the molecule; if a 217G variant is claimed, then only in connection with other, catalytically compensating point mutations in this substrate-binding loop. The only variants actually disclosed (p. 14) with replacements in positions 205 and/or 217 are those in which both residues have been replaced with space-filling, usually also aliphatic amino acids. The same applies to application WO 95/29979. When possessing a mutation in loop 6, the subtilisins of application WO 95/30010 have at least one further mutation in a different loop. Examples of 217G variants which are disclosed are Y217G/S188D or those with even more replacements in other loops than loop 6, whose homologous amino acids are unchanged in the variants of the invention.

Subtilisin BPN′ serves as reference enzyme of the subtilisins, in particular with respect to numbering of positions. Thus, for example, the point mutations of application EP 130756 which refer to all subtilisins are also indicated with BPN′ numbering. These also include position 217 which corresponds to position 211 in the enzyme of the invention. Further relevant positions have not been described previously by this document.

The publications by E. L. Smith et al. from 1968 in J. Biol. Chem., Volume 243, pp. 2184-2191 and by Jacobs et al. (1985), Nucl. Acids Res., Volume 13, pp. 8913-8926 introduce the protease subtilisin Carlsberg. It is naturally produced by Bacillus licheniformis and obtainable under the trade name Alcalase® from Novozymes A/S, Bagsværd, Denmark. Variants thereof which are obtainable by point mutations and have reduced binding to the substrate with a simultaneously increased rate of hydrolysis are disclosed, for example, by application WO 96/28566. As in the BPN′ applications discussed above, these are variants in which single or multiple exchanges in the loop regions of the molecule have been carried out; a variant 204I/216G (Carlsberg numbering) has not been described previously therein.

The PB92 protease is produced naturally by the alkaliphilic bacterium Bacillus nov. spec. 92 and obtainable under the trade name Maxacal® from Gist-Brocades, Delft, The Netherlands. Its original sequence is described in patent application EP 283075. Variants of said enzyme which have been obtained by point mutation and which are suitable for use in detergents and cleaning agents are disclosed in applications WO 94/02618 and EP 328229. From the first of these, only the variant L211G/N212D has a replacement identical to that of the variant claimed herein; no relevant variant emerges from the second application.

The subtilisins 147 and 309 are sold by Novozymes under the trade names Esperase® and Savinase®, respectively. They are derived from Bacillus clausii strains which are disclosed by application GB 1243784. Variants of said enzymes, which have been developed by means of point mutagenesis with respect to usage in detergents and cleaning agents are disclosed, for example, in applications WO 89/06279, WO 95/30011, WO 99/27082 and WO 00/37599.

Application WO 89/06279 aims at achieving higher oxidation stability, an increased rate of proteolysis and enhanced washing performance of the protease. It reveals (p. 14) that only replacements at particular positions should alter the physical or chemical properties of subtilisin 147 or 309 molecules; the positions 3, 4 and 211 are not included here. Application WO 95/30011 introduces variants of subtilisin 309 which have point mutations in the loop regions of the molecule and thus exhibit reduced adsorption to the substrate with a simultaneously increased rate of hydrolysis; they do not include any mutations in positions 3 or 4; the only point mutation actually corresponding to the variant of the present application is the L211G substitution which, however, does not correlate in any case with a V199I substitution. Application WO 99/27082 develops variants of, by way of example, subtilisin 309, whose washing performance is enhanced by enlarging the active loops by inserting two or more amino acids. Thus, they are not substitutions like in the present application.

Further examples of proteases established for use in detergents and cleaning agents are:
protease 164-A1 from Chemgen Corp., Gaithersburg, Md., USA, and Vista Chemical Company, Austin, Tex., USA (WO 93/07276), obtainable from Bacillus spec.;
Bacillus sp. PD138 NCIMB 40338 alkaline protease from Novozymes (WO 93/18140);
proteinase K-16 from Kao Corp., Tokyo, Japan (U.S. Pat. No. 5,344,770), derived from Bacillus sp. ferm. BP-3376;
subtilisin DY, described by Nedkov et al. 1985 in Biol. Chem Hoppe-Seyler, Volume 366, pp. 421-430, which has been optimized, in particular for usage in detergents and cleaning agents, by application WO 96/28557, again via specific point mutations in the active loops, but not including a V204I variant (corresponding to position 199 in the enzyme of the invention) either alone or in combination with other substitutions; and
thermitase produced by Thermoactinomyces vulgaris (Meloun et al., FEBS Lett. 1983, pp. 195-200) and optimized, for example, according to application WO 96/28558.

Among numerous possible variants of thermitase, the document mentioned last also describes the variants with the L221G substitution (corresponding to position 211 in the enzyme of the invention). Since the enzyme by nature has isoleucine at position 209 (corresponding to 199 in the enzyme of the invention), two of the amino acid residues important for the invention at the corresponding positions (corresponding to 199I/L211G) have hereby been previously described, but without the additional feature of additionally stabilizing the molecule in the presence of said two amino acids at said positions. In particular, no stabilizations by threonine in position 3 and/or isoleucine at position 4 (according to B. lentus alkaline protease) have been described previously. However, thermitase has the amino acid residues serine and arginine at positions 10 and 11 which are homologous to said two positions of B. lentus alkaline protease (compare alignment in WO 91/00345).

Moreover, thermitase is a molecule whose sequence over-all deviates considerably from those of the other subtilisins (Meloun et al., p. 198). Thus the homology between the mature proteins thermitase and B. lentus alkaline protease is 45% identity (62% similar amino acids). Something similar applies to proteinase K (WO 96/28556) whose homology to B. lentus alkaline protease is only 33% identity (46% similar amino acids) at the mature protein level.

The applications EP 199404, EP 251446, WO 91/06637 and WO 95/10591, for example, describe further proteases which are referred to by Procter & Gamble Comp., Cincinnati, Ohio, USA as “protease A”, “protease B”, “protease C” and “protease D”, respectively, and which may be used in detergents and cleaning agents (compare WO 00/47707, p. 73). The proteases of application EP 199404 are various variants which are based on patent EP 130756, but which have no variations at the positions relevant to the present application (compare EP 199404 A2, column 20). Example 10 of patent EP 251446 B1 (p.49) demonstrates that Y217G variants are less stable than the wild-type enzyme and, therefore, this substitution is not pursued any further. According to application WO 91/06637, “proteases C” are distinguished by point mutations at positions 123 and 274. According to WO 95/10591, all “protease D” variants carry mutations at position 76 which is unchanged in the present protease and identical to that of BPN′; the same applies to the application and, respectively, patents WO 95/10615, U.S. Pat. Nos. 6,017,871 and 6,066,611.

Other known proteases are the enzymes obtainable under the trade names Durazym®, Relase®, Everlase®, Nafizym, Natalase® and Kannase® from Novozymes, under the trade names Purafect®, Purafect OxP® and Properase® from Genencor, under the trade name Protosol® from Advanced Biochemicals Ltd., Thane, India and under the trade name Wuxi® from Wuxi Snyder Bioproducts Ltd., China.

In order to enhance the washing performance of subtilisins, numerous applications pursued the strategy of inserting additional amino acids into the active loops, thus, for example, apart from the applications already mentioned, also the applications published with the numbers WO 00/37599, WO 00/37621 to WO 00/37627 and WO 00/71683 to WO 00/71691.

Another strategy is to alter the surface charges of the molecule. Thus, for example, the applications WO 91/00334, WO 91/00335, WO 91/00345, EP 479870, EP 945502 and EP 563103, introduce numerous amino acid substitutions which can be used to increase or decrease the isoelectric point of said molecules. From this, application WO 00/24924 derives a method for identifying appropriately suitable variants. The same applies with respect to WO 96/34935 according to which it is also possible to vary the hydrophobicity of said molecules according to the same principle.

Another strategy for improving the washing performance of subtilisins is to randomly introduce point mutations into known molecules and to test the variants obtained for their contributions to the washing performance. This strategy is pursued, for example, by patent U.S. Pat. No. 5,700,676 in which the only position described which is relevant to the present invention is a substitution at position 217 (BPN′ numbering), in each case in addition to a plurality of other substitutions. The same also applies to patents U.S. Pat. Nos. 5,310,675, 5,801,038, 5,955,340 and applications WO 99/20723 and WO 99/20727. The only mutation proposed in patent U.S. Pat. No. 4,760,025, which is relevant to the present invention, is one at position 217, the reason for which is that said mutation affects the active site. All of these documents do not suggest that the other substitutions of the invention could play a part with respect to washing performance.

A modern direction in enzyme development is to combine, via statistical methods, elements from known proteins related to one another to novel enzymes having properties which have not been achieved previously. Methods of this kind are also summarized under the generic term directed evolution and include, for example, the following methods: The StEP method (Zhao et al. (1998), Nat. Biotechnol., Volume 16, p. 258-261), Random priming recombination (Shao et al., (1998), Nucleic Acids Res., Volume 26, p. 681-683), DNA shuffling (Stemmer, W. P. C. (1994), Nature, Volume 370, p. 389-391) or RACHITT (Coco, W. M. et al. (2001), Nat. Biotechnol., Volume 19, p. 354-359).

Another, in particular complimenting, strategy is to increase the stability of the proteases concerned and thus to increase their efficacy. For example, U.S. Pat. No. 5,230,891 has described a stabilization of this kind for proteases used in cosmetics. For detergents and cleaning agents, on the other hand, stabilizations by point mutations are more familiar. Thus, according to U.S. Pat. Nos. 6,087,315 and 6,110,884, proteases can be stabilized by replacing particular tyrosine residues with other residues. Other possibilities are, for example:
replacing particular amino acid residues with proline, according to EP 583339;
introducing more polar or charged groups on the molecule surface, according to EP 995801;
altering the binding of metal ions, in particular calcium binding sites, for example according to the teaching of applications WO 88/08028 and WO 88/08033;
   further possibilities of stabilizing subtilisins, in particular those derived from that of Bacillus lentus, are reported in patents U.S. Pat. Nos. 5,340,735, 5,500,364, 5,985,639 and 6136553.

The B. lentus alkaline proteases are highly alkaline proteases of Bacillus species. One of these strains has been deposited under number DSM 5483 (WO 91/02792, and, respectively, EP 493398 and U.S. Pat. No. 5,352,604). WO 92/21760, WO 95/23221 and WO 98/30669 disclose variants of this enzyme to be obtained by point mutation and usable in detergents and cleaning agents.

The wild-type enzyme is derived from a producer which had originally been obtained by screening for alkaliphilic Bacillus strains and displays itself a comparatively high stability to oxidation and the action of detergents. The applications WO 91/02792 and, respectively, EP 493398 and U.S. Pat. No. 5,352,604 describe its heterologous expression in the host Bacillus licheniformis ATCC 53926. The claims of said US patent refer to positions 208, 210, 212, 213 and 268, but not to any variant having substitutions in positions 61 and 211, as being characteristic for B. lentus alkaline protease.

Application WO 92/21760 also discloses the amino acid sequence, under SEQ ID NO:52, and the nucleotide sequence, under SEQ ID NO:106, of the B. lentus alkaline protease wild-type enzyme. In addition, this application discloses 51 different variants which differ from the wild-type in numerous positions, among them also S3T, V4I and V199I.

The applications WO 95/23221 and WO 98/30669 also reveal B. lentus alkaline protease variants suitable for usage in detergents and cleaning agents, which correspond to the enzyme of the invention in the three positions S3T, V4I and V199I. In addition, they all have two or three further point mutations compared to the wild-type enzyme from the B. lentus DSM 5483. Some of them carry an additional mutation at position 211, namely 211D (variants F49, F54 and F55); consequently, said applications claim the substitutions 211D and 211E.

As all of these studies which have been carried out over a long period of time confirm, there is high demand for alternative proteases for usage in detergents and cleaning agents. The most recent publications such as, for example, WO 00/71683 to WO 00/71691, prove that even the long established family of subtilisin proteases is still in need of optimization with respect to their usability in detergents and cleaning agents. Said need of optimization is accompanied by numerous studies on variation in the amino acid sequence of the enzymes concerned. However, the behavior of said enzymes in the context of a detergent or cleaning agent formulation cannot be readily inferred from the possibly calculatable enzymic properties (compare U.S. Pat. Nos. 5,801,039, 5,985,639 and 6,136,553). Other factors, such as stability to oxidizing agents, denaturation by surfactants, folding effects or desired synergies with other ingredients, play a part here.

It was the object of the present invention to find subtilisins which show improved performances in technical applications. In particular, it was intended to find those subtilisins which improve the washing or cleaning performance of detergents and/or cleaning agents.

Part of the object had been not only to improve the proteases with respect to their hydrolytic activity but also to maintain their stability in appropriate detergent and cleaning agent formulations.

With respect to this problem, the present patent application pursued the strategy of further improving the Bacillus lentus DSM 5483 subtilisin, in particular compared to the molecules disclosed in applications WO 91/02792, WO 92/21760 and WO 95/23221, for usage in detergents and cleaning agents.

Surprisingly, it was found that the amino acids isoleucine and glycine at positions 199 and 211 result in an increased washing performance contribution which is enhanced, presumably via a stabilizing effect, by the amino acids threonine and isoleucine at positions 3 and 4, respectively.

According to the invention, this object is thus achieved by alkaline proteases of the subtilisin type, which are characterized in that, according to the numbering of Bacillus lentus DSM 5483 subtilisin, they have isoleucine at position 199 and glycine at position 211 and at least one stabilization, preferably due to the amino acids threonine at position 3 and/or isoleucine at position 4.

It is likewise achieved by subtilisin variants which are characterized in that, according to the numbering of Bacillus lentus DSM 5483 subtilisin, they have isoleucine at position 199 and glycine at position 211 and, more preferably, additionally one or both of the amino acids threonine at position 3 and isoleucine at position 4.

It is likewise achieved by subtilisin variants which are characterized in that, according to the numbering of Bacillus lentus DSM 5483 subtilisin, they have threonine at position 3, isoleucine at position 4, isoleucine at position 199 and glycine at position 211.

It is particularly achieved by appropriate alkaline proteases of the subtilisin type which are characterized in that they are naturally produced by a bacillus or can be derived from such a subtilisin, in particular of Bacillus lentus.

Very particularly, it is achieved by alkaline proteases which are naturally produced by Bacillus lentus DSM 5483 or can be derived from such alkaline proteases, and among these in particular B. lentus alkaline protease S3T/V4I/V199I/L211G according to the amino acid sequence indicated in SEQ ID NO.4.

The B. lentus alkaline protease variant M131 with the characterizing substitutions S3T/V4I/A188P/V193M/V199I must be regarded as the variant from WO 92/21760, which has the highest degree of homology to the B. lentus alkaline protease variant of the invention, S3T/V4I/V199I/L211G. It corresponds in three positions to those of the variant of the invention. The difference is the two substitutions A188P and V193M at whose positions the variant of the invention is identical to the wild type. As, for example, application WO 95/30011 demonstrates, amino acid 193 of B. lentus subtilisins is located at the start of loop 6, while amino acid 188 is to be assigned not to any loop but to the compact protein region located in between. In this respect, both mutations are located in structurally different regions of the molecule. Surprisingly, it was found in. the present invention that reversing the two positions 188 and 193 to the wild-type amino acids and an additional mutation in position 211, i.e. in the posterior region of loop 6, results in an enzyme which is superior to the previously known enzymes, in particular the previously known variants of B.lentus alkaline protease, with respect to its washing and cleaning performance.

The particularly preferred enzyme of the invention differs from the variants of applications WO 95/23221 and WO 98/30669 in that a plurality of positions have reverted, i.e. are identical again to the wild type, and that position 211 contains the non-space-saving and uncharged amino acid glycine instead of the leucine of the wild type or the aspartate of said variants.

From an enzymological point of view, it is surprising that the effect of improved washing and cleaning performance is achieved by a substitution in an amino acid which is presumably involved in substrate binding and/or catalysis of the reaction; namely by replacing the space-saving, hydrophobic side chain of leucine with the side chain of a glycine, which is reduced to a proton. At the same time, the second position of loop 6, which had been mutated compared to the wild type, namely 199, need not be reverted from isoleucine to valine of the wild type. In comparison to the applications WO 95/23221 or WO 98/30669, the point mutagenesis to give an acidic group, i.e. L211D or L211E, would have been more obvious than reversion to the wild-type sequence at the other mutated positions. The documents, cited at the outset, for variation of the active loops of the various subtilisins, in particular WO 95/30011, would, with variation of position 211, have suggested an additional change in another active loop or a more drastic change within the same loop, such as, for example, V199S/L211D, P204E/L211G or G196S/L211G, in order to compensate for the change catalytically, but by no means sticking to the V199I substitution.

It is surprising, from the viewpoint of application, in particular in detergents and cleaning agents, that this results in performance improvement, in particular in an improvement of contribution of such enzymes to the washing and cleaning performance on a large variety of soilings. The successful use of subtilisins of the invention in appropriate washing and cleaning agent formulations (compare Examples 2 to 5) suggests that the stability of the variants concerned is also high enough in order to keep the enzymes active for a sufficiently long period and has thus contributed to improved performance.

The present invention relates to an alkaline protease of the subtilisin type, characterized in that, according to the numbering of Bacillus lentus DSM 5483 substilisin, it has isoleucine at position 199 and glycine at position 211 and at least one stabilization. Preferably, said stabilization is additionally one of the amino acids threonine at position 3 or isoleucine at position 4.

Further embodiments of this subject matter of the invention are alkaline proteases of the subtilisin type, characterized in that, according to the numbering of Bacillus lentus DSM 5483 subtilisin, they have threonine at position 3, isoleucine at position 4, isoleucine at position 199 and glycine at position 211; that they are subtilisins naturally produced by a Bacillus, in particular by Bacillus lentus, or derived from such a Bacillus; that they are subtilisins naturally produced by or derived from Bacillus lentus DSM 5483, in particular B. lentus alkaline protease S3T/V4I/V199I/L211G according to the amino acid sequence indicated in SEQ ID NO.4.

Further embodiments of this subject matter of the invention are proteins derived from corresponding alkaline proteases of the subtilisin type, in particular by fragmentation or deletion mutagenesis, by insertion mutagenesis, by substitution mutagenesis or by fusion of at least one part to at least one other protein; those additionally characterized in that they are additionally derivatized; that they have a proteolytic activity, preferably an increased proteolytic activity compared to the starting molecule and, respectively, nonderivatized molecule, and very particularly enhanced performance; and/or that they are additionally stabilized.

The invention further relates to nucleic acids which code for the proteins referred to in the first subject matter of the invention, in particular nucleic acids coding for subtilisin proteases, whose nucleotide sequence corresponds to the nucleotide sequence indicated in SEQ ID NO.3, in particular in the regions coding for 199 isoleucine and 211 glycine and very particularly in the regions coding for 3 threonine, 4 isoleucine, 199 isoleucine and 211 glycine.

The present invention further relates to vectors which contain a nucleic acid region as defined above and comprises, in particular, a nucleic acid region coding for any of the proteins or derivatives as defined in the first subject matter of the invention. They are, in preferred embodiments, cloning vectors which comprise a nucleic acid region as defined above and which comprise, in particular, a nucleic acid region coding for any of the proteins or derivatives as defined in the first subject matter of the invention; or they are expression vectors which comprise a nucleic acid region as defined above and which comprise, in particular, a nucleic acid region coding for any of the proteins or derivatives as defined in the first subject matter of the invention and making possible the biosynthesis thereof.

The invention further relates to cells which comprise a vector according to the abovementioned subject matter of the invention; which preferably express or can be induced to express any of the proteins or derivatives as defined in the first subject matter of the invention, in particular by using an expression vector as defined above; which are preferably characterized in that they are bacteria, in particular those which secrete the protein produced into the surrounding medium; which are preferably characterized in that they are bacteria of the genus Bacillus, in particular of the species Bacillus lentus, Bacillus licheniformis, Bacillus amyloliquefaciens, Bacillus subtilis or Bacillus alcalophilus; or which are characterized in that they are eukaryotic cells, in particular those which modify posttranslationally the produced protein.

The invention further relates to methods for preparing a proteolytic enzyme or derivative according to the first subject matter of the invention by using a host cell as defined above and/or using a vector as defined above and/or using a nucleic acid as defined above.

The invention further relates to agents which are characterized in that they comprise proteolytic enzymes according to the first subject matter of the invention, in particular detergents or cleaning agents, very particularly in an amount of from 2 μg to 20 mg per g of agent; preferably those which are characterized in that they additionally comprise further enzymes, in particular other proteases, amylases, cellulases, hemicellulases and/or lipases.

The invention further relates to agents for the treatment of textile raw materials or for textile care, which are characterized in that they contain either solely or in addition to other active ingredients, a proteolytic enzyme according to the first subject matter of the invention, in particular for fibers or textiles containing natural components and, very particularly, for those containing wool or silk.

The invention further relates to methods for machine cleaning textiles or hard surfaces, which methods are characterized in that in at least one of the method steps a proteolytic enzyme according to the first subject matter of the invention becomes active, preferably in an amount of from 40 μto 4 g, particularly preferably from 400 μto 400 mg, per application.

The invention further relates to methods for the treatment of textile raw materials or for textile care, which methods are characterized in that in at least one of the method steps a proteolytic enzyme according to the first subject matter of the invention becomes active, in particular for textile raw materials or textiles containing natural components, in particular for those containing wool or silk.

The invention further relates to uses of a proteolytic enzyme according to the first subject matter of the invention for cleaning textiles or hard surfaces, preferably in an amount of from 40 μto 4 g, particularly preferably from 400 μto 400 mg, per application.

The invention further relates to uses of a proteolytic enzyme according to the first subject matter of the invention for activating or deactivating ingredients of detergents or cleaning agents.

The invention further relates to uses of a proteolytic enzyme according to the first subject matter of the invention for biochemically analyzing or for synthesizing low molecular weight compounds or proteins.

The invention further relates to uses of a proteolytic enzyme according to the first subject matter of the invention for preparing, purifying or synthesizing natural substances or biological valuable substances.

The invention further relates to uses of a proteolytic enzyme according to the first subject matter of the invention for the treatment of natural raw materials, in particular for the treatment of surfaces, very particularly in a method for the treatment of leather.

The invention further relates to uses of a proteolytic enzyme according to a the first subject matter of the invention for the obtainment or treatment of raw materials or intermediates in the manufacture of textiles, in particular for removing protective layers on fabrics.

The invention further relates to uses of a proteolytic enzyme according to the first subject matter of the invention for the treatment of textile raw materials or for textile care, in particular for the treatment of wool or silk or of wool- or silk-containing mixed textiles.

The invention further relates to uses of a proteolytic enzyme according to the first subject matter of the invention for the treatment of photographic films, in particular for removing gelatin-containing or similar protective layers.

The invention further relates to uses of a proteolytic enzyme according to the first subject matter of the invention for preparing food or animal feed.

The invention further relates to cosmetics containing a proteolytic enzyme according to the first subject matter of the invention or to cosmetic methods including a proteolytic enzyme according to the first subject matter of the invention or to the use of a proteolytic enzyme according to the first subject matter of the invention for cosmetic purposes, in particular within the framework of corresponding methods or in corresponding agents.

A protein means in accordance with the present application a polymer which is composed of the natural amino acids, has a substantially linear structure and adopts usually a three-dimensional structure to exert its function. Table 1 lists the 19 proteinogenic, naturally occurring L-amino acids, together with the 1- and 3-letter codes which are also used in the present application for abbreviation of said amino acids.

TABLE 1
The proteinogenic amino acids

| 1-letter code | 3-letter code | Full name |
|---|---|---|
| A | Ala | alanine |
| C | Cys | cysteine |
| D | Asp | aspartic acid |
| E | Glu | glutamic acid |
| F | Phe | phenylalanine |
| G | Gly | glycine |
| H | His | histidine |
| I | Ile | isoleucine |
| K | Lys | lysine |
| L | Leu | leucine |
| M | Met | methionine |
| N | Asn | asparagine |
| P | Pro | proline |
| Q | Gln | glutamine |
| R | Arg | arginine |
| S | Ser | serine |
| T | Thr | threonine |
| V | Val | Valine |
| W | Trp | tryptophane |

The combination of any of these names/codes with a number indicates the amino acid residue which the particular protein carries at the respective position. Thus, for example, S3 indicates a serine residue at position 3, starting with the numbering at the N terminus of the protein in question. According to this nomenclature, a point mutation at this site, for example to give the amino acid threonine, is abbreviated with S3T. In order to denote variants having a plurality of point mutations, these substitutions are separated from one another by forward slashes. Accordingly, the variant S3T/V4I is characterized in that the serine previously present at position 3 of said variant has been replaced with a threonine and the valine at position 4 has been replaced with an isoleucine.

Unless stated otherwise, the positions indicated in the present invention refer to the in each case mature forms of the proteins concerned, i.e. without the signal peptides (see below).

An enzyme in accordance with the present application means a protein which exerts a particular biochemical function. Proteolytic enzymes or enzymes with proteolytic function, for example, mean generally those which hydrolyze the acid amide bonds of proteins, in particular those bonds located inside the proteins, and which may therefore also be referred to as endopeptidases. Subtilisin proteases are those endopeptidases which are naturally produced by Gram-positive bacteria and usually secreted or which are derived from the latter, for example via molecular biological methods, and can be homologized with the natural subtilisin proteases via part regions such as structure-forming or function-carrying regions. They are described, for example, in the article “Subtilases: Subtilisin-like Proteases” by R. Siezen, pages 75-95 in “Subtilisin enzymes”, edited by R. Bott and C. Betzel, New York, 1996.

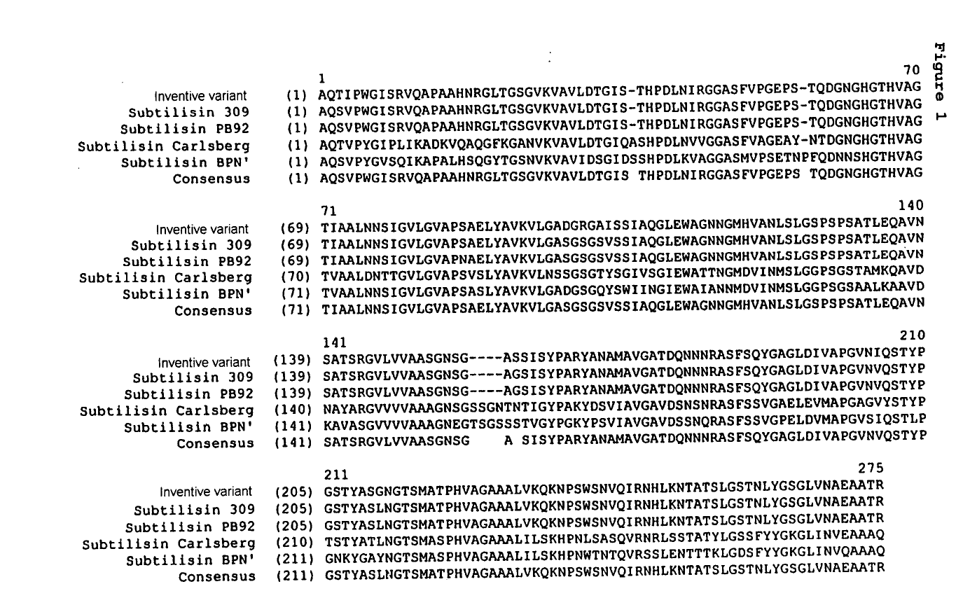

Numerous proteins are formed as “preproteins”, i.e. together with a signal peptide. This then means the N-terminal part of the protein, whose function usually is to ensure the export of the produced protein from the producing cell into the periplasm or into the surrounding medium and/or the correct folding thereof. Subsequently, the signal peptide is removed from the remaining protein under natural conditions by a signal peptidase so that said protein exerts its actual catalytic activity without the initially present N-terminal amino acids. According to FIG. 1 in WO 91/02792, the preprotein of Bacillus lentus DSM 5483 subtilisin contains 380 amino acids, the mature protein, however, only 269; the numbering starts with the first amino acid of the mature protein, i.e. in this case with the alanine which would have number 112 according to the preprotein sequence. According to SEQ ID NO. 1 and 2, the signal peptide of B. licheniformis ATCC 68614 subtilisin is 111 amino acids and the mature peptide 269 amino acids in length. Without this division, the complete protein is 380 amino acids in length, as SEQ ID NO.2 reveals. According to SEQ ID NO.3 and 4, the same applies to the particularly preferred embodiment.

Owing to their enzymic activity, preference is given for technical applications to the mature peptides, i.e. the enzymes processed after their preparation, over the preproteins.

Pro-proteins are inactive precursors of proteins. Their precursors with signal sequence are referred to as prepro-proteins.

Nucleic acids mean in accordance with the present application the molecules which are naturally composed of nucleotides, serve as information carriers and code for the linear amino acid sequence in proteins or enzymes. They may be present as single strand, as a single strand complementary to said single strand or as double strand. For molecular-biological work, preference is given to the nucleic acid DNA as the naturally more durable information carrier. In contrast, an RNA is produced to implement the invention in a natural environment such as, for example, in an expressing cell, and RNA molecules important to the invention are therefore likewise embodiments of the present invention.

In accordance with the present application, the information unit of a nucleic acid, which corresponds to a protein, is also referred to as gene. In the case of DNA, the sequences of both complementary strands in in each case all three possible reading frames must be taken into account. The fact that different codon triplets can code for the same amino acids so that a particular amino acid sequence can be derived from a plurality of different nucleotide sequences which possibly have only low identity must also be taken into account (degeneracy of the genetic code). Moreover, various organisms differ in the use of these codons. For these reasons, both amino acid sequences and nucleotide sequences must be incorporated into the scope of protection, and nucleotide sequences indicated are in each case regarded only as coding by way of example for a particular amino acid sequence.

It is possible for a skilled worker, via nowadays generally known methods such as, for example, chemical synthesis or polymerase chain reaction (PCR) in combination with molecular-biological and/or protein-chemical standard methods, to prepare complete genes on the basis of known DNA sequences and/or amino acid sequences. An ideal starting point for this are DNA preparations of deposited and/or commercially available microorganisms. Such methods are known, for example, from the “Lexikon der Biochemie [Encyclopedia of Biochemistry]”, Spektrum Akademischer Verlag, Berlin, 1999, Volume 1, pp. 267-271 and Volume 2, pp. 227-229.

Changes of the nucleotide sequence, as may be produced, for example, by molecular-biological methods known per se, are referred to as mutations. Depending on the type of change, deletion, insertion or substitution mutations, for example, or those in which various genes or parts of genes are fused to one another (shuffling) are known; these are gene mutations. The corresponding organisms are referred to as mutants. The proteins derived from mutated nucleic acids are referred to as variants. Thus, for example, deletion, insertion, substitution mutations or fusions result in deletion-, insertion-, substitution-mutated or fusion genes and, at the protein level, in corresponding deletion, insertion or substitution variants, or fusion proteins.

Vectors mean in accordance with the present invention elements which consist of nucleic acids and which contain a gene of interest as characteristic nucleic acid region. They are capable of establishing said gene as a stable genetic element replicating independently of the remaining genome in a species or a cell line over several generations or cell divisions. Vectors are, in particular when used in bacteria, special plasmids, i.e. circular genetic elements. Genetic engineering distinguishes between, on the one hand, those vectors which are used for storage and thus, to a certain extent, also for genetic engineering work, the “cloning vectors”, and, on the other hand, those which perform the function of establishing the gene of interest in the host cell, i.e. enabling expression of the protein in question. These vectors are referred to as expression vectors.

Homologization, i.e. comparison with known enzymes, as carried out via an alignment, for example, makes it possible to deduce the enzymic activity of an enzyme studied from the amino acid or nucleotide sequence. Said activity may be modified qualitatively or quantitatively by other regions of the protein which are not involved in the actual reaction. This could concern, for example, enzyme stability, activity, reaction conditions or substrate specificity.

The term proteolytic enzyme or protease therefore means, in addition to the functions of the few amino acid residues of the catalytically active site, any functions as resulting from the action of the entire remaining protein or one or more parts of the remaining protein on the actually catalytically active regions. In accordance with the invention, such modifying functions or part activities alone are also regarded as proteolytic activity, as long as they support a proteolytic reaction. Such auxiliary functions or part activities include, for example, binding of a substrate, an intermediate or an end product, the activation or inhibition or mediation of a regulating influence on the hydrolytic activity. Another possible example is the formation of a structural element located far away from the active site. The second precondition for the fact that it is a protein of the invention, however, is that the chemical behavior of the actually active residues alone or, in addition, the action of the modifying parts results in a hydrolysis of peptide bonds. It is furthermore possible that one or more parts of, for example, the protein of the invention also modify qualitatively or quantitatively the activities of other proteases. This influencing of other factors is regarded as proteolytic activity. Proteolytically active enzymes are also those whose activity at a given point in time is blocked, for example by an inhibitor. Their principal suitability for the corresponding proteolytic reaction is crucial.

Fragments mean any proteins or peptides which are smaller than natural proteins or those which correspond to completely translated genes, and may also be obtained synthetically, for example. Owing to their amino acid sequences, they may be related to the corresponding complete proteins. They may adopt, for example, identical structures or exert proteolytic activities or partial activities such as complexing of a substrate, for example. Fragments and deletion variants of starting proteins are in principle very similar; while fragments represent rather relatively small pieces, the deletion mutants rather lack only short regions and thus only individual partial functions.

Chimeric or hybrid proteins mean in accordance with the present application those proteins which are composed of elements which naturally originate from different polypeptide chains from the same organism or from different organisms. This procedure is also called shuffling or fusion nmtagenesis. The purpose of such a fusion may be, for example, to cause or to modify an enzymic function with the aid of the fused-to protein part of the invention. In accordance with the present invention, it is unimportant as to whether such a chimeric protein consists of a single polypeptide chain or of a plurality of subunits between which different functions may be distributed. To implement the latter alternative, it is possible, for example, to break down a single chimeric polypeptide chain into a plurality of polypeptide chains by a specific proteolytic cleavage, either posttranslationally or only after a purification step.

Proteins obtained by insertion mutation mean those variants which have been obtained via methods known per se by inserting a nucleic acid fragment or protein fragment into the starting sequences. They should be classified as chimeric proteins, due to their similarity in principle. They differ from the latter merely in the size ratio of the unaltered protein part to the size of the entire protein. In such insertion-mutated proteins the proportion of foreign protein is lower than in chimeric proteins.

Inversion mutagenesis, i.e. a partial sequence conversion, may be regarded as a special form of both deletion and insertion. The same applies to a regrouping of various molecule parts, which deviates from the original amino acid sequence. Said regrouping can be regarded as deletion variant, as insertion variant and as shuffling variant of the original protein.

Derivatives mean in accordance with the present application those proteins whose pure amino acid chain has been chemically modified. Those derivatizations may be carried out, for example, biologically in connection with protein biosynthesis by the host organism. Molecular-biological methods may be employed here. However, said derivatizations may also be carried out chemically, for example by chemical conversion of an amino acid side chain or by covalent binding of another compound to the protein. Such a compound may also be, for example, other proteins which are bound, for example, via bifunctional chemical compounds to proteins of the invention. Such modifications may influence, for example, substrate specificity or the strength of binding to the substrate or cause transient blocking of the enzymic activity if the coupled-to substance is an inhibitor. This may be useful for the period of storage, for example. Likewise, derivatization means covalent binding to a macromolecular support.

In accordance with the present invention, all enzymes, proteins, fragments and derivatives, unless they need to be explicitly referred to as such, are included under the generic term proteins.

The performance of an enzyme means its efficacy in the technical area considered in each case. Said performance is based on the actual enzymic activity but, in addition, depends on further factors relevant for the particular process. These include, for example, stability, substrate binding, interaction with the material carrying said substrate or interactions with other ingredients, in particular synergies.

The washing or cleaning performance of an agent means in accordance with the present application the effect exerted by the agent studied on the soiled articles, for example textiles or objects with hard surfaces. Individual components of such agents, for example individual enzymes, are evaluated with respect to their contribution to the washing or cleaning performance of the entire agent, for it is not readily possible to deduce the contribution of an enzyme to the washing performance of an agent from the enzymic properties of said enzyme. Examples of other factors which play a part here are stability, substrate binding, binding to the material to be cleaned and interactions with other ingredients of said agents, in particular synergies in removing the soilings.

According to the Budapest Treaty on the international recognition of the deposit of microorganisms from Apr. 28, 1977, the following microorganism has been deposited in connection with application WO 91/02792, on Aug. 10, 1989, with the Deutsche Sammlung von Mikroorganismen und Zellkulturen GmbH in Brunswick, Germany: Bacillus lentus DSM 5483. There it has the registration number DSM 5483 (PA5-A0155, Strain 2-1). The essential information on the features of this biological material is summarized in WO 91/02792, Table 1 (pages 5 to 7). The DNA sequence and amino acid sequence of the alkaline protease from said organism, which is particularly relevant to the present application, can be found under SEQ ID NO:106 and SEQ ID NO:52, respectively.

Particularly important to the invention are positions 3, 4, 199 and 211 of the mature proteins according to the Bacillus lentus DSM 5483 subtilisin numbering (WO 92/21760). These can be homologized according to Table 2 with those of the most important subtilisins; said homologization can be transferred to all other subtilisins. Thus, for example, the article “Subtilases: Subtilisin-like Proteases” by R. Siezen, pages 75-95 in “Subtilisin enzymes”, edited by R. Bott and C. Betzel, New York, 1996 shows an alignment of more than 20 subtilisins in relation to the known sequence of subtilisin BPN′.

TABLE 2
Homologization of the four positions
particularly important to the invention

| | Numbering | | | | |
|---|---|---|---|---|---|
| Reference | according to the | | | Pos. | Pos. |
| enzymes | sequences in | Pos. 3 | Pos. 4 | 199 | 211 |
| B. lentus | WO 92/21760 | S 3 | V 4 | V 199 | L 211 |
| alkaline | | | | | |
| protease | | | | | |
| BPN′ | Wells et al. (see | S 3 | V 4 | I 205 | Y 217 |
| | above) | | | | |
| Subtilisin | Smith et al. (see | T 3 | V 4 | V 204 | L 216 |
| Carlsberg | above) | | | | |
| PB92 | EP 283075 | S 3 | V 4 | V 199 | L 211 |
| Subtilisin | WO 89/06279 | S 3 | V 4 | V 199 | L 211 |
| 309 | | | | | |
| Thermitase | WO 91/00345 | S 10 | R 11 | I 209 | L 221 |
| Proteinase K | WO 91/00345 | T 4 | A 6 | I 208 | I 220 |

FIG. 1 of the present patent application also depicts an alignment of the amino acid sequences of a B. lentus alkaline protease variant of the invention with these most important subtilisins described at the outset, namely Subtilisin 309 (Savinase®), Subtilisin PB92, Subtilisin Carlsberg and Subtilisin BPN′.

Owing to the high structural homologies between the various known subtilisins and to the same reaction mechanism of hydrolyzing endogenous acid amide bonds, which is exerted by them, it can be expected that said point mutations act in each case comparably in the context of the molecule in question. In particular, it can be expected, owing to the teaching of the present patent application, that such subtilisins which have already been developed in the prior art with regard to their usage in detergents and cleaning agents are improved further with respect to their contributions to the washing and cleaning performances by adopting these point mutations.

Adopting the amino acids isoleucine 199 and glycine 211 at the homologous positions, in particular, should contribute to improving the contribution of enzymes known from the prior art to the washing and cleaning performance in appropriate agents. Owing to the experiences with B.lentus alkaline protease, however, these substitutions will profit from at least one additional stabilization of the molecules concerned.

The stability of inventive proteases having the amino acid positions 199I and 211G may be increased, for example, by coupling to polymers. Such a method is described in U.S. Pat. No. 5,230,891, for example. It requires linking the proteins, prior to their use in appropriate agents, via a chemical coupling step to such polymers.

Preference is given to stabilizations possible via point mutagenesis of the molecule itself, since they do not require any further working steps following obtainment of the protein. Some point mutations suitable for this are known per se from the prior art. Thus, according to U.S. Pat. Nos. 6,087,315 and 6,110,884, proteases may be stabilized by replacing particular tyrosine residues with other residues. Applied to Bacillus lentus-derived proteins of the invention, this would mean substitutions of the tyrosine residues at positions 89, 161, 165, 208 and 257, according to SEQ ID NO.2; the other two positions indicated there are already occupied by tyrosine anyway in B. lentus alkaline protease.

Other Possibilities Are, for Example:

replacing particular amino acid residues with proline, according to EP 583339; this would mean for enzymes derived from B.lentus the substitutions S55P, A96P, A166P, A188P and/or S253P);
introducing more polar or charged groups on the surface of the molecule, according to EP 995801;
altering the binding of metal ions, in particular the calcium binding sites, for example according to the teaching of applications WO 88/08028 and WO 88/08033. According to the first of these documents, one or more of the amino acid residues involved in calcium binding should be replaced with negatively charged amino acids. According to the teaching of the second document, point mutations should be introduced simultaneously in at least one of the sequences of the two residues arginine/glycine; this relates, for example in Bacillus lentus subtilisins, to the NG sequences in positions 60/61, 115/116 and 212/213.
According to U.S. Pat. No. 5,453,372, proteins may be protected by particular mutations on the surface against the effect of denaturing agents such as surfactants; the positions indicated there correspond to positions 134, 155, 158, 164, 188 and/or 189 in B. lentus alkaline protease. Further comparable possibilities are indicated in U.S. Pat. Nos. 5,340,735, 5,500,364, 5,985,639 and 6,136,553.

In preferred embodiments, stabilization occurs due to the amino acid residues of threonine at position 3 and/or of isoleucine at position 4, according to the Bacillus lentus DSM 5483 subtilisin numbering.

The variants studied in the examples of the present invention suggest that their increased stability is the decisive factor for imparting to them an improved washing performance, acting together with amino acids 199I and 211G.

Independently of this theory, all alkaline proteases of the subtilisin type which are characterized in that, according to the numbering of Bacillus lentus DSM 5483 subtilisin, they have isoleucine at position 199 and glycine at position 211 and additionally have either of the two amino acids threonine at position 3 and isoleucin at position 4 are solutions to the object of the invention.

In addition, preference is given to those variants which have, in addition to 199 isoleucine and 211 glycine, both threonine at position 3 and isoleucine at position 4.

Preference is given to corresponding variants of those alkaline proteases naturally produced by or derived from a Bacillus, since Bacillus proteases have from the outset properties advantageous for various possible technical uses, including a certain stability to a temperature, oxidizing or denaturing agents. Moreover, most experience has been obtained with microbial proteases, with respect to their biotechnological production regarding, for example, construction of suitable cloning vectors, selection of host cells and fermentation conditions or evaluation of risks such as, for example, allergenicity.

Very particularly established in the prior art are the subtilisins of Bacillus lentus and the subtilisins derived from its naturally produced proteases, for example for use in detergents and cleaning agents. They include the proteases mentioned at the outset, subtilisin 147, subtilisin 309 and B.lentus alkaline protease. The wealth of experience acquired for preparation and use of said proteases benefits further developments of said enzymes according to the invention, including, for example, their compatibility with other chemical compounds such as, for example, the ingredients of detergents or cleaning agents.

A particularly preferred embodiment relates to the proteases of the invention, which can be derived from those produced by Bacillus lentus DSM 5483. Included here are, for example, those of the variants described in applications WO 92/21760 and WO 95/23221 and WO 98/30669. Developments of these enzymes, which have the substitutions of the invention at positions 199, 211, 3 and/or 4, characterize particularly preferred embodiments of the present invention.

The B.lentus alkaline protease S3T/V4I/V199I/L211G studied in the present application is a development of B. lentus DSM 5483 subtilisin whose amino acid sequence is disclosed under SEQ ID NO:52 and whose nucleotide sequence is disclosed under SEQ ID NO:106 in the sequence listing of application WO 92/21760.

It was found that the contribution of this novel variant to an agent corresponding to the washing and cleaning performance was higher than that of the comparable enzymes B.lentus alkaline protease F49 and Savinase® established in the prior art for these purposes (Examples 2-5). The sequence listing lists the amino acid sequence of this variant under number SEQ ID NO.2. The gene coding for this amino acid sequence is listed in the sequence listing under number SEQ ID NO. 1. Owing to the degeneracy of the genetic codes, numerous other nucleic acids are also conceivable which likewise code for said variant and are equally preferred alternatives within this subject matter of the invention.

A bacteria, in particular Bacillus, strain which produces the B.lentus alkaline protease S3T/V4I/V199I/L211G variant with the DNA sequences and amino acid sequences indicated in the sequence listing may be prepared, for example, following the method illustrated in Example 1 of the present application.

Further proteins can be derived via molecular-biological methods established in the prior art from the alkaline proteases mentioned previously. Such methods are also discussed in detail in the textbook Fritsch, Sambrook and Maniatis “Molecular cloning: a laboratory manual”, Cold Spring Harbour Laboratory Press, New York, 1989, for example.

Said further proteins include, for example, variants to which additional properties have been imparted via substitution mutagenesis or via further point mutations and which are, due to said additional properties predestined with respect to specific possible uses, for example due to changes in surface charges, as disclosed in WO 00/36069, or due to alterations in the loops involved in catalysis or substrate binding, as disclosed in WO 99/27082, for example. It is also possible to subject larger partial regions of said variants to mutagenesis. Thus it may be the aim of fragment generation or deletion mutagenesis, for example, to select specific partial functions of the protease or, on the other hand, to exclude them, for example substrate binding and the interactions with other compounds, exerted via particular regions of the molecule.

Insertion, substitution or fusion may provide proteases of the invention with additional functions. This includes possibly, for example, coupling to particular domains, such as binding to cellulose-binding domains, as described in the publications WO 99/57154 to WO 99/57159, for example. The amino acid linkers denoted here may be constructed by forming an integrated fusion protein of protease, linker region and binding domain. Such a binding domain could also come from the same or a different protease, for example in order to enhance binding of the protein of the invention to a protease substrate. This increases the local protease concentration, which increase may be advantageous in individual applications, for example in the treatment of raw materials.

Said proteases may also be derivatized, in particular for optimizing them for their particular target application. This includes chemical modifications, as described, for example, in application DE 40 13 142. They may also be modified, for example, by coupling of low or high molecular weight chemical compounds, as are carried out by nature in connection with protein biosynthesis by various organisms, such as, for example, binding of a fatty acid radical close to the N terminus or glycosylations in synthesis by eukaryotic host cells. Proteolytic enzymes or fragments which are additionally derivatized are thus embodiments of the present invention.

In connection with the use of proteins of the invention in detergents or cleaning agents, coupling to other detersive substances or enzymes, for example, is particularly useful. The patent applications WO 00/18865 and WO 00/57155, for example, describe comparable coupling approaches for cellulose-binding domains. Analogously, couplings to macromolecular compounds such as, for example, polyethylene glycol may also be carried out in order to modify the molecule with respect to further properties such as stability or skin compatibility. U.S. Pat. No. 5230891, for example, describes a modification of this kind for rendering the proteases in question more suitable for the use in cosmetics.

Derivatives of proteins of the invention can, in the broadest sense, also mean preparations of these enzymes. Depending on its obtainment, working-up or preparation, a protein may be associated with various other substances, for example from the culture of the producing microorganisms, since culture supernatants of protease-producing microorganisms already exhibit a proteolytic activity, indicating that even crude extracts may be used appropriately, for example for inactivating other proteinogenic activities.

A protein may also have been specifically admixed with particular other substances, for example to increase its storage stability. Therefore, any preparations of the actual protein of the invention are also in accordance with the invention. This is also independent of whether or not it actually produces said enzymic activity in a particular preparation, since it may be desired that it has only low activity, if any, during storage and produces its proteolytic function only when used. This may depend, for example, on the folding state of the protein or may result from the reversible binding of one or more accompanying substances of the preparation to a protein of the invention. The joint preparation of proteases with protease inhibitors, in particular, is known from the prior art (WO 00/01826). Also included here are fusion proteins in which the inhibitors are bound via linkers, in particular amino acid linkers, to the particular proteases (WO 00/01831).

Said developments, derivatizations and preparations of proteins of the invention are particularly desired if said proteins continue to be proteolytically active, since this is the precondition for their possible uses of the invention. Preferably, the proteases obtained by any kind of mutagenesis and/or derivatization have, compared to the starting molecule and the non-derivatized molecule, respectively, increased proteolytic activity and very particularly improved performances with respect to their in each case intended technical field of use, including, in particular, improvement of their washing and/or cleaning performance for use in detergents or cleaning agents.

This is possible, for example, by combining the point mutations of the invention with further point mutations which relate to the catalytic reaction, for example at the active site. Thus it would be possible, following the teaching of application WO 95/30011, for example, to mutate proteases of the invention which are those derived from Bacillus lentus subtilisin, in the loop regions or to introduce additional amino acids. Such studies are described in the applications published under numbers WO 00/37599, WO 00/37621 to WO 00/37627 and WO 00/71683 to WO 00/71691.

The deletion of a region of the enzyme, which interacts with other active compounds in the reaction medium and thus impairs the overall reaction, for example via folding effects, could be such a desired development. Analogously, fusion to other active enzymes, for example to other proteases, is conceivable in order to achieve an increased rate of hydrolysis.

The reversible blocking of a proteolytic activity during storage, due to binding of an inhibitor, for example, can stop autoproteolysis and thus effect a high rate of proteolysis in the reaction medium at the time of dilution. Coupling to special binding domains, for example, may increase in the purification process the concentration of the protease close to the substrate relative to that in the liquor and thus increase the contribution of said enzyme to the performance of the agent.

Numerous possibilities of increasing the stability of enzymes, in particular of those used in detergents and cleaning agents, are known from the prior art (see above). Particularly relevant to the invention among these are, for practicability reasons, those methods which are based on point mutagenesis. All of the possibilities already illustrated above can also be applied in combination to variants of the invention, since, according to WO 89/09819, it can be assumed that multiple stabilizing mutations have an additive effect. Thus, variants of the invention which have already been stabilized by either of or both of the two amino acids 3T and 4I, can be additionally stabilized by coupling to a polymer. They may, however, also have a stabilizing mutation at a different site of the molecule, for example due to substitution of one or more of the tyrosine residues defined above, introduction of particular proline residues, alteration of surface charges or alteration of calcium-binding sites.

Nucleic acids are the starting point for virtually all common molecular-biological studies and developments of proteins and production thereof, including, in particular, sequencing of the genes and derivation of the corresponding amino acid sequence, any type of mutagenesis and expression of the proteins. As already mentioned above, such methods are described, for example, in the manual by Fritsch, Sambrook and Maniatis “Molecular cloning: a laboratory manual”, Cold Spring Harbour Laboratory Press, New York, 1989. The second subject matter of the invention are therefore nucleic acids coding for the proteins of the first subject matter of the invention or for derivatives thereof.

At the DNA level, the enzymes important to the invention may be optimized for various applications via any methods generally listed under the term “protein engineering”. This makes it possible, in particular, to achieve the following properties which occur at the protein level: improvement of the resistance of the derived protein to oxidation, of the stability to denaturing agents or proteases, to high temperatures, to acidic or strongly alkaline conditions, alteration of the sensitivity to calcium or other cofactors, reduction in immunogenicity or allergenic action.

Examples of mutated genes of the invention include those responsible for individual, specific base substitutions or randomized point mutations, for deletions of individual bases or of partial sequences, fusions to other genes or gene fragments or inversions. Mutations or modifications of this kind can predestine the enzyme derived from the respective nucleic acids for specific applications. Such a mutagenesis may be carried out target-specifically or via random methods, for example using a subsequent recognition and/or selection method (screening and selection) on the cloned genes, targeting the activity.

In particular for those nucleic acids coding for protein fragments, all three reading frames, both in sense and in antisense orientation, must be taken into account, since such oligonucleotides can be used via the polymerase chain reaction (PCR) as starting points for the synthesis of related nucleic related acids. Such oligonucleotides are explicitly included within the scope of protection of the present invention, in particular when covering any of the regions corresponding to the four amino acid positions 3, 4, 199 and/or 211. This applies also to those which have variable sequences in exactly these positions so that, within a population of many primers, there may also be at least one that codes for a partial sequence corresponding to SEQ ID NO.3 for such a position. The same applies to antisense oligonucleotides which may be used for regulating expression, for example.

The development of the proteases of the invention may be orientated in particular on the ideas presented in the publication “Protein engineering” by P. N. Bryan (2000) in Biochim. Biophys. Acta, Volume 1543, pp. 203-222.

Preference is given to nucleic acids coding for subtilisin proteases, whose nucleotide sequence corresponds to the nucleotide sequence indicated in SEQ ID NO.3. This applies particularly for the regions coding for 199 isoleucine and 211 glycine and very particularly for those coding for 3 threonine, 4 isoleucine, 199 isoleucine and 211 glycine.

This preferentially applies to those which can be derived from a sequence for a Bacillus lentus protease, and particularly, if they can be derived from a sequence for a Bacillus lentus DSM 5483 protease. In a very particularly preferred case, the nucleic acid codes for the B.lentus alkaline protease S3T/V4I/V199I/L211G of the invention and/or corresponds to the nucleotide sequence indicated in SEQ ID NO.3.

The scope of protection also includes, for example, those nucleic acids coding for proteolytically active insertion or fusion mutants. Thus the region responsible for this activity may be fused, for example, to cellulose-binding domains or may carry point mutations in catalytically inactive regions in order to enable the derived protein to be coupled to a polymer or to reduce the allergenicity thereof.

In order to handle the nucleic acids relevant to the invention, they are conveniently ligated into vectors. This includes, for example, vectors derived from bacterial plasmids, from viruses or bacterial phages, or largely synthetic vectors. They are suitable starting points for molecular-biological and biochemical studies of the gene in question, of its expression or of the corresponding protein. Thus vectors containing the nucleic acid molecules as defined above, in particular those coding for the proteolytic enzymes as defined above are a subject matter of the present invention.

Cloning vectors are preferred embodiments of said subject matter of the invention and are, in addition to storage, biological amplification or selection of the gene of interest, suitable for molecular-biological characterization of said gene. At the same time, they are transportable and storable forms of the claimed nucleic acids and are also starting points for molecular-biological techniques not linked to cells such as, for example, PCR or in-vitro mutagenesis methods. Preference is given to those cloning vectors containing nucleic acid regions coding for the proteolytic enzymes as defined above.

Expression vectors of the invention are the basis for implementing the nucleic acids of the second subject matter of the invention in biological production systems and thereby producing the proteins of the first subject matter of the invention. Preferred embodiments of said subject matter of the invention are expression vectors which carry all the genetic elements necessary for expression, for example the natural promoter originally located upstream of said gene or a promoter from another organism. Said elements may be arranged, for example, in the form of an “expression cassette”. Preference is given to those expression vectors comprising nucleic acid regions coding for the proteolytic enzymes as defined above.

Another possibility of implementing the present invention is cells containing a vector according to the third subject matter of the invention. They are thus the microbiological dimension of the present invention by making possible, for example, amplification of the appropriate genes but also mutagenesis or transcription and translation thereof and, ultimately, biotechnological production.

Host cells which express or can be induced to express any of the proteins of the invention, thereby enabling biotechnological production thereof, are an embodiment of said subject matter of the invention. For this purpose, they must have received, i.e. must have been transformed with, the appropriate gene, conveniently via a vector. Said vector may be present in the host cell extrachromosomally as separate genetic element or may have been integrated into a chromosome and is preferably any of the expression vectors as defined above.

Suitable host cells are in principle all organisms, i.e. prokaryotes, eukaryotes or Cyanophyta. Preference is given to those host cells which are easily manageable genetically, with respect to, for example, transformation with the expression vector and to its stable establishment, for example unicellular fungi or bacteria. Moreover, preferred host cells are distinguished by good microbiological and biotechnological manageability. This relates, for example, to easy culturability, high growth rates, low requirements on fermentation media and good rates of production and secretion of foreign proteins. Frequently, it is necessary to determine experimentally the expression systems optimal for the individual case from the abundance of various systems available according to the prior art. In this way, any protein of the invention can be obtained from a multiplicity of host organisms.

Preferred embodiments are those host cells whose activity can be regulated, owing to appropriate genetic elements, for example by controlled addition of chemical compounds, by changing the culturing conditions or as a function of the particular cell density. This controllable expression makes possible a very economical production of the proteins of interest.

In a preferred embodiment, the host cells are bacteria, in particular those which secrete the protein produced into the surrounding medium, since bacteria distinguish themselves by short generation times and low demands on the culturing conditions. This makes it possible to establish cost-effective methods. Gram-negative bacteria such as, for example, E. coli, secrete a multiplicity of proteins into the periplasmic space. This may be advantageous for special applications. In contrast, Gram-positive bacteria such as, for example, bacilli release secreted proteins immediately into the nutrient medium surrounding the cells, from which the expressed proteins of the invention can be purified directly, according to another preferred embodiment. Application WO 01/81597 even discloses a method according to which it is achieved that Gram-negative bacteria also export the expressed proteins.

One embodiment of the present invention utilizes Bacillus lentus DSM 5483 itself in order to (homologously) express proteins of the invention. On the other hand, however, preference is given to heterologous expression. Bacteria preferred for heterologous expression include those of the genus Bacillus, in particular those of the species Bacillus licheniformis, Bacillus amyloliquefaciens, Bacillus subtilis or other species or strains of Bacillus alcalophilus, since these produce comparable subtilisins themselves so that it is possible to obtain via this method a mixture of proteins of the invention with the subtilisins endogenously produced by the host strains. Application WO 91/02792 (EP 493398 B1) describes, for example, coexpression of this kind of B.lentus alkaline protease in Bacillus licheniformis ATCC 53926; numerous possible expression vectors can also be found there. It can be expected that the newly found variants of the invention, in particular those of Bacillus lentus and very particularly B.lentus alkaline protease S3T/V4I/V199I/L211G can be prepared with the aid of said vectors and/or in said host system.

In another preferred embodiment of this subject matter of the invention, the host cells are eukaryotes, in particular those which modify the produced protein posttranslationally. Examples of suitable eukaryotes are fungi such as actinomycetes or yeasts such as Saccharomyces or Kluyveromyces. The modifications which such systems carry out, in particular in connection with protein synthesis, include binding of low molecular weight compounds such as membrane anchors or oligosaccharides, for example. Oligosaccharide modifications of this kind may be desirable for reducing the allergenicity of the prepared proteins, for example.

Methods for preparing a proteolytic enzyme or derivative of the invention are a separate subject matter of the invention. Thus it is possible, for example on the basis of the above-defined DNA sequences and amino acid sequences, as can be derived, for example, also from the sequence listing, to synthesize corresponding oligopeptides and oligonucleotides up to the complete genes and proteins according to molecular-biological methods known per se. Starting from the known subtilisin-producing microorganisms, it is also possible to isolate further natural subtilisin producers, to determine their subtilisin sequences and to develop them further, according to the guidelines devised herein. Bacterial species of this kind may also be cultured and used for appropriate production methods. Analogously, novel expression vectors can be developed according to the model of the vectors disclosed in application WO 91/02792, for example. Cell-free expression systems in which protein biosynthesis is carried out in vitro may also be embodiments of the present invention, on the basis of the corresponding nucleic acid sequences. Any elements already set forth above may also be combined to give novel methods in order to prepare proteins of the invention. In this connection, a multiplicity of possible combinations of method steps for each protein of the invention is conceivable so that the optimal method should be determined experimentally for each specific individual case.

Agents characterized in that they contain a proteolytic enzyme of the invention are a separate subject matter of the invention.

Virtually all possible technical uses of enzymes of the invention depend on using the functional enzyme in an appropriate medium. Thus, for example, the possible microbiological uses demand agents in which the enzyme, usually in the form of highly pure preparations, is combined with the necessary reaction partners or cofactors. Agents for the treatment of raw materials or cosmetic preparations are likewise characterized by specific formulations. According to the invention, all these formulations should be understood as being agents containing the enzyme of the invention.

Preferred embodiments included in this subject matter of the invention are detergents or cleaning agents since, as the exemplary embodiments of the present application show, it was surprisingly found that a subtilisin variant 199I/211G (numbering according to B.lentus alkaline protease) or a Bacillus lentus variant having the substitutions S3T/V4I/V199I/L211G, in particular the B.lentus alkaline protease S3T/V4I/V199I/L211G variant derived from Bacillus lentus DSM 5483, gives a distinct performance increase on various soilings (compare Examples 2 and 3), which exceeds the level of established detergent proteases with the same amount of activity used. The same applies to corresponding mechanical dishwasher agents (compare Examples 4 and 5). This effect occurs reproducibly both at different temperatures and at different concentrations.

This subject matter of the invention includes any conceivable types of cleaning agents, both concentrates and agents to be applied in undiluted form; for use on the commercial scale, in the washing machine or for manual laundry or cleaning. They include, for example, detergents for textiles, carpets or natural fibers, for which the term detergent is used in the present invention. They also include, for example, dishwashing agents for dishwashers or manual dishwashing agents or cleaners for hard surfaces such as metal, glass, porcelain, ceramic, tiles, stone, coated surfaces, plastics, wood or leather; for those, the term cleaning agent is used in the present invention. Any type of cleaning agent is an embodiment of the present invention, as long as a protein of the invention has been added to it.

Embodiments of the present invention comprise any presentations of the agents of the invention, which are established in the prior art and/or appropriate. They include, for example, solid, pulverulent, liquid, gel-like or paste-like agents, where appropriate also composed of a plurality of phases, compressed or uncompressed; further examples include: extrudates, granules, tablets or pouches, packaged both in large containers and in portions.

Agents of the invention contain enzymes of the invention in an amount of from 2 μg to 20 mg and, increasingly preferably, from 5 μg to 17.5 mg, from 20 μg to 15 mg, from 50 μg to 10 mg, from 100 μg to 7.5 mg, from 200 μg to 5 mg and from 500 μg to 1 mg, per gram of agent. This results in amounts of from 40 μg to 4 g and, increasingly preferably, from 50 μg to 3 g, from 100 μg to 2 g, from 200 μg to 1 g and, particularly preferably, from 400 μg to 400 mg per application.

The protease activity in agents of this kind may be determined according to the method described in Tenside, Volume 7 (1970), pp. 125-132 and is, accordingly, indicated in protease units (PE=Protease-Einheiten). The protease activity of the agents may be up to 1,500,000 protease units per gram of preparation.

Apart from an enzyme important to the invention, an agent of the invention contains, where appropriate, further ingredients such as surfactants, for example nonionic, anionic and/or amphoteric surfactants, and/or bleaches, and/or builders, and, where appropriate, further conventional ingredients.

The nonionic surfactants used are preferably alkoxylated, advantageously ethoxylated, in particular primary alcohols having preferably from 8 to 18 carbon atoms and, on average, from 1 to 12 mol of ethylene oxide (EO) per mole of alcohol, in which the alcohol radical can be linear or, preferably, methyl-branched in the 2-position or can comprise linear and methyl-branched radicals in a mixture as are customarily present in oxo alcohol radicals. Particular preference is, however, given to alcohol ethoxylates containing linear radicals of alcohols of native origin having from 12 to 18 carbon atoms, for example from coconut, palm, tallow fatty or oleyl alcohol, and, on average, from 2 to 8 EO per mole of alcohol. Preferred ethoxylated alcohols include, for example, C₁₂₋₁₄-alcohols having 3 EO or 4 EO, C₉₋₁₁-alcohol having 7 EO, C₁₃₋₁₅-alcohols having 3 EO, 5 EO, 7 EO or 8 EO, C₁₂₋₁₈-alcohols having 3 EO, 5 EO or 7 EO, and mixtures of these, such as mixtures of C₁₂₋₁₄-alcohol having 3 EO and C₁₂₋₁₈-alcohol having 5 EO. The degrees of ethoxylation given are statistical averages which may be an integer or a fraction for a specific product. Preferred alcohol ethoxylates have a narrowed homolog distribution (narrow range ethoxylates, NRE). In addition to these nonionic surfactants, fatty alcohols having more than 12 EO can also be used. Examples thereof are tallow fatty alcohol having 14 EO, 25 EO, 30 EO or 40 EO.

A further class of preferably used nonionic surfactants which are used either as the sole nonionic surfactant or in combination with other nonionic surfactants are alkoxylated preferably ethoxylated or ethoxylated and propoxylated fatty acid alkyl esters, preferably having from 1 to 4 carbon atoms in the alkyl chain, in particular fatty acid methyl esters.

A further class of nonionic surfactants which can advantageously be used are the alkyl polyglycosides (APG). Alkyl polyglycosides which may be used satisfy the general formula RO(G)_{z}, in which R is a linear or branched, in particular methyl-branched in the 2-position, saturated or unsaturated, aliphatic radical having from 8 to 22, preferably from 12 to 18 carbon atoms, and G is the symbol which stands for a glycose unit having 5 or 6 carbon atoms, preferably for glucose. The degree of glycosylation z is here between 1.0 and 4.0, preferably between 1.0 and 2.0 and in particular between 1.1 and 1.4. Preference is given to using linear alkyl polyglucosides, i.e. alkyl polyglycosides in which the polyglycosyl radical is a glucose radical, and the alkyl radical is an n-alkyl radical.

Nonionic surfactants of the amine oxide type, for example N-cocoalkyl-N,N-dimethylamine oxide and N-tallow alkyl-N,N-dihydroxyethylamine oxide, and of the fatty acid alkanolamides may also be suitable. The proportion of these nonionic surfactants is preferably no more than that of the ethoxylated fatty alcohols, in particular no more than half thereof.

Further suitable surfactants are polyhydroxy fatty acid amides of the formula (II) 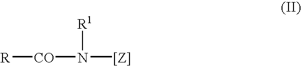
in which RCO is an aliphatic acyl radical having from 6 to 22 carbon atoms, R¹ is hydrogen, an alkyl or hydroxyalkyl radical having from 1 to 4 carbon atoms and [Z] is a linear or branched polyhydroxyalkyl radical having from 3 to 10 carbon atoms and from 3 to 10 hydroxyl groups. The polyhydroxy fatty acid amides are known substances which can usually be obtained by reductive amination of a reducing sugar with ammonia, an alkylamine or an alkanolamine and subsequent acylation with a fatty acid, a fatty acid alkyl ester or a fatty acid chloride.

The group of polyhydroxy fatty acid amides also includes compounds of the formula (III) 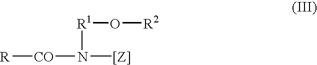
in which R is a linear or branched alkyl or alkenyl radical having from 7 to 12 carbon atoms, R¹ is a linear, branched or cyclic alkyl radical or an aryl radical having from 2 to 8 carbon atoms, and R² is a linear, branched or cyclic alkyl radical or an aryl radical or an oxy-alkyl radical having from 1 to 8 carbon atoms, where C₁₋₄-alkyl or phenyl radicals are preferred, and [Z] is a linear polyhydroxyalkyl radical whose alkyl chain is substituted with at least two hydroxyl groups, or alkoxylated, preferably ethoxylated or propoxylated, derivatives of this radical.

[Z] is preferably obtained by reductive amination of a reducing sugar, for example glucose, fructose, maltose, lactose, galactose, mannose or xylose. The N-alkoxy- or N-aryloxy-substituted compounds may be converted, for example by reaction with fatty acid methyl esters in the presence of an alkoxide as catalyst, into the desired polyhydroxy fatty acid amides.

The anionic surfactants used are, for example, those of the sulfonate and sulfate type. Suitable surfactants of the sulfonate type are preferably C₉₋₁₃-alkylbenzene sulfonates, olefin sulfonates, i.e. mixtures of alkene and hydroxyalkane sulfonates, and disulfonates, as obtained, for example, from C₁₂₋₁₈-monoolefins having a terminal or internal double bond by sulfonation with gaseous sulfur trioxide and subsequent alkaline or acidic hydrolysis of the sulfonation products. Also suitable are alkane sulfonates which are obtained from C₁₂₋₁₈-alkanes, for example, by sulfochlorination or sulfoxidation with subsequent hydrolysis or neutralization. Likewise suitable are also the esters of α-sulfo fatty acids (estersulfonates), for example the α-sulfonated methyl esters of hydrogenated coconut, palm kernel or tallow fatty acids.

Further suitable anionic surfactants are sulfated fatty acid glycerol esters. Fatty acid glycerol esters mean the mono-, di- and triesters, and mixtures thereof, as are obtained during the preparation by esterification of a monoglycerol with from 1 to 3 mol of fatty acid or during the transesterification of triglycerides with from 0.3 to 2 mol of glycerol. Preferred sulfated fatty acid glycerol esters are here the sulfation products of saturated fatty acids having from 6 to 22 carbon atoms, for example of capronic acid, caprylic acid, capric acid, myristic acid, lauric acid, palmitic acid, stearic acid or behenic acid.

Preferred alk(en)yl sulfates are the alkali metal, and in particular the sodium, salts of sulfuric half-esters of C₁₂-C₁₈-fatty alcohols, for example of coconut fatty alcohol, tallow fatty alcohol, lauryl, myristyl, cetyl or stearyl alcohol or of C₁₀-C₂₀-oxo alcohols and those half-esters of secondary alcohols of these chain lengths. Further preferred are alk(en)yl sulfates of said chain length which comprise a synthetic, petrochemical-based straight-chain alkyl radical which have analogous degradation behavior to the equivalent compounds based on fatty chemical raw materials. From a washing performance viewpoint, preference is given to C₁₂-C₁₆-alkyl sulfates and C₁₂-C₁₅-alkyl sulfates, and C₁₄-C₁₅-alkyl sulfates. 2,3-Alkyl sulfates are also suitable anionic surfactants.

The sulfuric monoesters of straight-chain or branched C₇₋₂₁-alcohols ethoxylated with from 1 to 6 mol of ethylene oxide, such as 2-methyl-branched C₉₋₁₁-alcohols having, on average, 3.5 mol of ethylene oxide (EO) or C₁₂₋₁₈-fatty alcohols having from 1 to 4 EO, are also suitable. Owing to their high foaming behavior, they are used in cleaning agents only in relatively small amounts, for example in amounts up to 5% by weight, usually from 1 to 5% by weight.

Further suitable anionic surfactants are also the salts of alkylsulfosuccinic acid, which are also referred to as sulfosuccinates or as sulfosuccinic esters and which are monoesters and/or diesters of sulfosuccinic acids with alcohols, preferably fatty alcohols and, in particular, ethoxylated fatty alcohols. Preferred sulfosuccinates contain C₈₋₁₈-fatty alcohol radicals or mixtures thereof. Particularly preferred sulfosuccinates contain a fatty alcohol radical derived from ethoxylated fatty alcohols, which are themselves nonionic surfactants (see above for description). In this connection, sulfosuccinates whose fatty alcohol radicals are derived from ethoxylated fatty alcohols having a narrowed homolog distribution are, in turn, particularly preferred. Likewise, it is also possible to use alk(en)ylsuccinic acid having preferably from 8 to 18 carbon atoms in the alk(en)yl chain or salts thereof.

Further suitable anionic surfactants are, in particular, soaps. Saturated fatty acid soaps such as the salts of lauric acid, myristic acid, palmitic acid, stearic acid, hydrogenated erucic acid and behenic acid, and, in particular, soap mixtures derived from natural fatty acids, for example coconut, palm kernel or tallow fatty acids, are suitable.

The anionic surfactants including soaps may be present in the form of their sodium, potassium or ammonium salts, and as soluble salts of organic bases such as mono-, di- or triethanolamine. The anionic surfactants are preferably in the form of their sodium or potassium salts, in particular in the form of the sodium salts.

The surfactants may be present in the cleaning agents or detergents of the invention in an overall amount of from preferably 5% by weight to 50% by weight, in particular from 8% by weight to 30% by weight, based on the finished agent.

Agents of the invention may contain bleaches. Of the compounds which serve as bleaches and produce H₂O₂ in water, sodium percarbonate, sodium perborate tetrahydrate and sodium perborate monohydrate are of particular importance. Other bleaches which can be used are, for example, peroxopyrophosphates, citrate perhydrates and H₂O₂-producing peracidic salts or peracids, such as persulfates or persulfuric acid. Also useful is the urea peroxohydrate percarbamide which can be described by the formula H₂N—CO—NH₂.H₂O₂. In particular when used for cleaning hard surfaces, for example for machine dishwashing, the agents, if desired, may also contain bleaches from the group of organic bleaches, although the use thereof is possible in principle also in agents for washing textiles. Typical organic bleaches are diacyl peroxides such as, for example, dibenzoyl peroxide. Further typical organic bleaches are the peroxy acids, specific examples being alkyl peroxy acids and aryl peroxy acids. Preferred representatives are peroxy benzoic acid and its ring-substituted derivatives, such as alkylperoxybenzoic acids, but also peroxy-α-naphthoic acid and magnesium monoperphthalate, the aliphatic or substituted aliphatic peroxy acids such as peroxylauric acid, peroxystearic acid, ε-phthalimidoperoxycaproic acid (phthalimidoperoxyhexanoic acid, PAP), o-carboxy-benzamidoperoxycaproic acid, N-nonenylamidoperadipic acid and N-nonenylamidopersuccinate, and aliphatic and araliphatic peroxydicarboxylic acids such as 1,12-diperoxycarboxylic acid, 1,9-diperoxyazelaic acid, diperoxysebacic acid, diperoxybrassylic acid, diperoxyphthalic acids, 2-decyldiperoxybutane-1,4-dioic acid, N,N-terephthaloyl-di(6-aminopercaproic acid) may be used.

The bleach content of the agents may be from 1 to 40% by weight and, in particular, from 10 to 20% by weight, using advantageously perborate monohydrate or percarbonate.

In order to achieve improved bleaching action in cases of washing at temperatures of 60° C. and below, and in particular in the case of laundry pretreatment, the agents may also include bleach activators. Bleach activators which can be used are compounds which, under perhydrolysis conditions, give aliphatic peroxocarboxylic acids having preferably from 1 to 10 carbon atoms, in particular from 2 to 4 carbon atoms, and/or substituted or unsubstituted perbenzoic acid. Substances which carry O- and/or N-acyl groups of said number of carbon atoms and/or substituted or unsubstituted benzoyl groups are suitable. Preference is given to plurally acylated alkylenediamines, in particular tetraacetylethylenediamine (TAED), acylated triazine derivatives, in particular 1,5-diacetyl-2,4-dioxohexahydro-1,3,5-triazine (DADHT), acylated glycoluriles, in particular 1,3,4,6-tetraacetylglycoluril (TAGU), N-acylimides, in particular N-nonanoylsuccinimide (NOSI), acylated phenol sulfonates, in particular n-nonanoyl- or isononanoyloxybenzene sulfonate (n- or iso-NOBS), acylated hydroxycarboxylic acids such as triethyl-O-acetyl citrate (TEOC), carboxylic anhydrides, in particular phthalic anhydride, isatoic anhydride and/or succinic anhydride, carboxamides such as N-methyldiacetamide, glycolide, acylated polyhydric alcohols, in particular triacetin, ethylene glycol diacetate, isopropenyl acetate, 2,5-diacetoxy-2,5-dihydrofuran and the enol esters disclosed in German patent applications DE 196 16 693 and DE 196 16 767, and acetylated sorbitol and mannitol, or mixtures thereof described in European patent application EP 0 525 239 (SORMAN), acylated sugar derivatives, in particular pentaacetylglucose (PAG), pentaacetylfructose, tetraacetylxylose and octaacetyllactose, and acetylated, optionally N-alkylated glucamine or gluconolactone, triazole or triazole derivatives and/or particulate caprolactams and/or caprolactam derivatives, preferably N-acylated lactams, for example N-benzoylcaprolactam and N-acetylcaprolactam, which are disclosed in international patent applications WO 94/27970, WO 94/28102, WO 94/28103, WO 95/00626, WO 95/14759 and WO 95/17498. The hydrophilically substituted acyl acetals disclosed in German patent application DE 196 16 769 and the acyl lactams described in German patent application DE 196 16 770 and in international patent application WO 95/14075 are likewise used with preference. It is also possible to use the combinations of conventional bleach activators disclosed in German patent application DE 44 43 177. Nitrile derivatives such as cyanopyridines, nitrile quats, e.g. N-alkylammoniumacetonitriles, and/or cyanamide derivatives may also be used. Preferred bleach activators are sodium 4-(octanoyloxy)benzenesulfonate, n-nonanoyl- or isononanoyloxybenzenesulfonate (n- or iso-NOBS), undecenoyloxybenzenesulfonate (UDOBS), sodium dodecanoyloxybenzenesulfonate (DOBS), decanoyloxybenzoic acid (DOBA, OBC 10) and/or dodecanoyloxybenzenesulfonate (OBS 12), and N-methylmorpholinium acetonitrile (MMA). Such bleach activators may be present in the customary quantitative range from 0.01 to 20% by weight, preferably in amounts from 0.1 to 15% by weight, in particular 1% by weight to 10% by weight, based on the total composition.

In addition to the conventional bleach activators or instead of them, it is also possible for “bleach catalysts” to be present. These substances are bleach-enhancing transition metal salts or transition metal complexes such as, for example, Mn, Fe, Co, Ru or Mo salene complexes or carbonyl complexes. Mn, Fe, Co, Ru, Mo, Ti, V and Cu complexes containing N-containing tripod ligands, and Co, Fe, Cu and Ru ammine complexes are also suitable as bleach catalysts, preference being given to using those compounds described in DE 197 09 284 A1. Acetonitrile derivatives, according to WO 99/63038, and bleach-activating transition metal complex compounds, according to WO 99/63041 are capable of developing a bleach-activating action in combination with amylases.

Agents of the invention usually contain one or more builders, in particular zeolites, silicates, carbonates, organic cobuilders and, where no ecological reasons oppose their use, also phosphates. The latter are the preferred builders for use in particular in cleaning agents for machine dishwashing.

Compounds which may be mentioned here are crystalline, layered sodium silicates of the general formula NaMSiₓO₂ₓ₊₁.yH₂O, where M is sodium or hydrogen, x is a number from 1.6 to 4, preferably from 1.9 to 4.0, and y is a number from 0 to 20, and preferred values for x are 2, 3 or 4. Crystalline phyllosilicates of this kind are described, for example, in European patent application EP 0 164 514. Preferred crystalline phyllosilicates of the formula indicated are those where M is sodium and x adopts the values 2 or 3. In particular, both β- and δ-sodium disilicates Na₂Si₂O₅.yH₂O are preferred. Compounds of this kind are sold, for example, under the name SKS® (Clariant). Thus, SKS-6® is primarily a δ-sodium disilicate having the formula Na₂Si₂O₅.yH₂O, and SKS-7® is primarily the β-sodium disilicate. Reacting the δ-sodium disilicate with acids (for example citric acid or carboxylic acid) gives kanemite NaHSi₂O₅.yH₂O, sold under the names SKS-9® and, respectively, SKS-10® (Clariant). It may also be advantageous to use chemical modifications of said phyllosilicates. The alkalinity of the phyllosilicates, for example, can thus be suitably influenced. Phyllosilicates doped with phosphate or with carbonate have, compared to the δ-sodium disilicate, altered crystal morphologies, dissolve more rapidly and display an increased calcium binding ability, compared to δ-sodium disilicate. Thus, phyllosilicates of the general empirical formula xNa₂O.ySiO₂O.zP₂O₅ where the x-to-y ratio corresponds to a number from 0.35 to 0.6, the x-to-z ratio to a number from 1.75 to 1 200 and the y-to-z ratio to a number from 4 to 2 800 are described in patent application DE 196 01 063. The solubility of the phyllosilicates may also be increased by using particularly finely granulated phyllosilicates. It is also possible to use compounds of the crystalline phyllosilicates with other ingredients. Compounds which may be mentioned here are in particular those with cellulose derivatives which have advantageous disintegrating action and are used in particular in detergent tablets, and those with polycarboxylates, for example citric acid, or polymeric polycarboxylates, for example copolymers of acrylic acid.

It is also possible to use amorphous sodium silicates having an Na₂O:SiO₂ modulus of from 1:2 to 1:3.3, preferably from 1:2 to 1:2.8, and in particular from 1:2 to 1:2.6, which have delayed dissolution and secondary detergent properties. The dissolution delay relative to conventional amorphous sodium silicates can have been induced by various means, for example by surface treatment, compounding, compaction/compression or by overdrying. Within the scope of this invention, the term “amorphous” also means “X-ray amorphous”. This means that in X-ray diffraction experiments the silicates do not give the sharp X-ray refractions typical of crystalline substances, but instead, at best, one or more maxima of these scattered X-rays, which have a width of several degree units of the diffraction angle. However, particularly good builder properties will very likely result if, in electron diffraction experiments, the silicate particles give poorly defined or even sharp diffraction maxima. This is to be interpreted to the effect that the products have microcrystalline regions with a size from 10 to a few hundred nm, preference being given to values up to at most 50 nm and in particular up to at most 20 nm. Particular preference is given to compressed/compacted amorphous silicates, compounded amorphous silicates and overdried X-ray amorphous silicates.

A finely crystalline, synthetic zeolite containing bonded water, which may be used where appropriate, is preferably zeolite A and/or P. As zeolite P, zeolite MAP® (commercial product from Crosfield) is particularly preferred. However, zeolite X and mixtures of A, X and/or P are also suitable. A product which is commercially available and can be used with preference within the scope of the present invention is, for example, also a co-crystallizate of zeolite X and zeolite A (approx. 80% by weight zeolite X), which is sold by CONDEA Augusta S.p.A. under the trade name VEGOBOND AX® and can be described by the formula
nNa₂O.(1−n)K₂O.Al₂O₃.(2-2.5)SiO₂.(3.5-5.5)H₂O

Suitable zeolites have an average particle size of less than 10 μm (volume distribution; measurement method: Coulter counter) and preferably contain from 18 to 22% by weight, in particular from 20 to 22% by weight, of bonded water.

Use of the generally known phosphates as builder substances is of course also possible, provided such a use should not be avoided for ecological reasons. Among the multiplicity of commercially available phosphates, the alkali metal phosphates are the most important in the detergents and cleaning agents industry, with pentasodium or pentapotassium triphosphate (sodium or potassium tripolyphosphate) being particularly preferred.

In this connection, alkali metal phosphates is the collective term for the alkali metal (in particular sodium and potassium) salts of the various phosphoric acids, it being possible to differentiate between metaphosphoric acids (HPO₃)ₙ and orthophosphoric acid H₃PO₄ as well as higher molecular weight representatives. The phosphates combine several advantages: they act as alkali carriers, prevent lime deposits on machine parts and lime incrustations in fabrics and, moreover, contribute to the cleaning performance.

Sodium dihydrogenphosphate, NaH₂PO₄, exists as dihydrate (density 1.91 gcm⁻³, melting point 60° C.) and as monohydrate (density 2.04 gcm⁻³). Both salts are white powders which are very readily soluble in water and which lose their water of crystallization upon heating and at 200° C. convert to the weakly acidic diphosphate (disodium hydrogendiphosphate, Na₂H₂P₂O₇), at a higher temperature to sodium trimetaphosphate (Na₃P₃O₉) and Maddrell's salt (see below). NaH₂PO₄ is acidic; it forms when phosphoric acid is adjusted to a pH of 4.5 using sodium hydroxide solution and the suspension is sprayed. Potassium dihydrogenphosphate (primary or monobasic potassium phosphate, potassium biphosphate, KDP), KH₂PO₄, is a white salt of density 2.33 gcm⁻³, has a melting point of 253° [decomposition with the formation of potassium polyphosphate (KPO₃)ₓ] and is readily soluble in water.

Disodium hydrogenphosphate (secondary sodium phosphate) Na₂HPO₄, is a colorless crystalline salt which is very readily soluble in water. It exists in anhydrous form and with 2 mol (density 2.066 gcm⁻³, loss of water at 95° C.), 7 mol (density 1.68 gcm⁻³, melting point 48° C. with loss of 5 H₂O) and 12 mol (density 1.52 gcm⁻³, melting point 35° C. with loss of 5 H₂O) of water, becomes anhydrous at 100° C. and upon more vigorous heating converts to the diphosphate Na₄P₂O₇. Disodium hydrogenphosphate is prepared by neutralizing phosphoric acid with soda solution using phenolphthalein as indicator. Dipotassium hydrogenphosphate (secondary or dibasic potassium phosphate), K₂HPO₄, is an amorphous, white salt which is readily soluble in water.

Trisodium phosphate, tertiary sodium phosphate, Na₃PO₄, are colorless crystals which, in the form of the dodecahydrate, have a density of 1.62 gcm⁻³ and a melting point of 73-76° C. (decomposition), in the form of the decahydrate (corresponding to 19-20% P₂O₅) have a melting point of 100° C. and in anhydrous form (corresponding to 39-40% P₂O₅) have a density of 2.536 gcm⁻³. Trisodium phosphate is readily soluble in water with an alkaline reaction and is prepared by evaporating a solution of exactly 1 mol of disodium phosphate and 1 mol of NaOH. Tripotassium phosphate (tertiary or tribasic potassium phosphate), K₃PO₄, is a white, deliquescent granular powder of density 2.56 gcm⁻³, has a melting point of 1 340° C. and is readily soluble in water with an alkaline reaction. It is produced, for example, during the heating of Thomas slag with carbon and potassium sulfate. Despite the higher price, the more readily soluble, and therefore highly effective, potassium phosphates are often preferred over corresponding sodium compounds in the cleaning agents industry.

Tetrasodium diphosphate (sodium pyrophosphate), Na₄P₂O₇, exists in anhydrous form (density 2.534 gcm⁻³, melting point 988° C., also 880° C. given) and as decahydrate (density 1.815-1.836 gcm⁻³, melting point 94° C. with loss of water). Both substances are colorless crystals which dissolve in water with an alkaline reaction. Na₄P₂O₇ is formed during the heating of disodium phosphate to >200° C. or by reacting phosphoric acid with soda in a stoichiometric ratio and dewatering the solution by spraying. The decahydrate complexes heavy metal salts and hardness constituents and thus reduces the water hardness. Potassium diphosphate (potassium pyrophosphate), K₄P₂O₇, exists in the form of the trihydrate and is a colorless, hygroscopic powder of density 2.33 gcm⁻³, which is soluble in water, the pH of the 1% strength solution at 25° C. being 10.4.

Condensation of NaH₂PO₄ and KH₂PO₄ results in higher molecular weight sodium phosphates and potassium phosphates, respectively, amongst which cyclic representatives, the sodium and potassium metaphosphates, respectively, and chain-shaped types, the sodium and potassium polyphosphates, respectively, can be differentiated. Particularly for the latter, a multiplicity of names are in use: melt or thermal phosphates, Graham's salt, Kurrol's and Maddrell's salt. All higher sodium and potassium phosphates are together referred to as condensed phosphates.

The industrially important pentasodium triphosphate, Na₅P₃O₁₀ (sodium tripolyphosphate), is a nonhygroscopic, white, water-soluble salt which is anhydrous or crystallizes with 6 H₂O and is of the general formula NaO-[P(O)(ONa)-O]ₙ-Na where n=3. In 100 g of water, about 17 g of the salt which is free of water of crystallization dissolve at room temperature, approx. 20 g dissolve at 60° C., and about 32 g dissolve at 100° C.; if the solution is heated at 100° C. for two hours, about 8% of orthophosphate and 15% of diphosphate form due to hydrolysis. In the preparation of pentasodium triphosphate, phosphoric acid is reacted with soda solution or sodium hydroxide solution in a stoichiometric ratio, and the solution is dewatered by spraying. Similarly to Graham's salt and sodium diphosphate, pentasodium triphosphate dissolves many insoluble metal compounds (including lime soaps, etc.). Pentapotassium triphosphate, K₅P₃O₁₀ (potassium tripolyphosphate), is available commercially, for example, in the form of a 50% strength by weight solution (>23% P₂O₅, 25% K₂O). The potassium polyphosphates are used widely in the detergents and cleaning agents industry. In addition, sodium potassium tripolyphosphates also exist which can likewise be used within the scope of the present invention. These form, for example, when sodium trimetaphosphate is hydrolyzed with KOH:
(NaPO₃)₃+2 KOH→Na₃K₂P₃O₁₀+H₂O

According to the invention, these can be used exactly as sodium tripolyphosphate, potassium tripolyphosphate or mixtures of these two; mixtures of sodium tripolyphosphate and sodium potassium tripolyphosphate or mixtures of potassium tripolyphosphate and sodium potassium tripolyphosphate or mixtures of sodium tripolyphosphate and potassium tripolyphosphate and sodium potassium tripolyphosphate can also be used according to the invention.

Organic cobuilders which can be used in the detergents and cleaning agents of the invention are, in particular, polycarboxylates or polycarboxylic acids, polymeric polycarboxylates, polyaspartic acid, polyacetals, optionally oxidized dextrins, further organic cobuilders (see below), and phosphonates. These classes of substance are described below.

Useable organic builder substances are, for example, the polycarboxylic acids usable in the form of their sodium salts, the term polycarboxylic acids meaning those carboxylic acids which carry more than one acid function. Examples of these are citric acid, adipic acid, succinic acid, glutaric acid, malic acid, tartaric acid, maleic acid, fumaric acid, sugar acids, aminocarboxylic acids, nitrilotriacetic acids (NTA), as long as such a use should not be avoided for ecological reasons, and mixtures thereof. Preferred salts are the salts of the polycarboxylic acids such as citric acid, adipic acid, succinic acid, glutaric acid, tartaric acid, sugar acids, and mixtures thereof.

It is also possible to use the acids per se. In addition to their builder action, the acids typically also have the property of an acidifying component and thus also serve to establish a lower and milder pH of detergents or cleaning agents, as long as the pH resulting from the mixture of the remaining components is not desired. Particular mention should be made here of environmentally safe acids such as citric acid, acetic acid, tartaric acid, malic acid, lactic acid, glycolic acid, succinic acid, glutaric acid, adipic acid, gluconic acid and any mixtures thereof. However, mineral acids, in particular sulfuric acid, or bases, in particular ammonium or alkali metal hydroxides, may also serve as pH regulators. The agents of the invention contain such regulators in amounts of preferably not more than 20% by weight, in particular from 1.2% by weight to 17% by weight.

Suitable builders are also polymeric polycarboxylates; these are, for example, the alkali metal salts of polyacrylic acid or of polymethacrylic acid, for example those having a relative molecular mass of from 500 to 70 000 g/mol.

The molar masses given for polymeric polycarboxylates are, for the purposes of this specification, weight-average molar masses, M_{W}, of the respective acid form, determined in principle by means of gel permeation chromatography (GPC), using a UV detector. The measurement was made against an external polyacrylic acid standard which, owing to its structural similarity toward the polymers studied, provides realistic molecular weight values. These figures differ considerably from the molecular weight values obtained using polystyrenesulfonic acids as the standard. The molar masses measured against polystyrenesulfonic acids are usually considerably higher than the molar masses given in this specification.

Suitable polymers are, in particular, polyacrylates which preferably have a molecular mass of from 2 000 to 20 000 g/mol. Owing to their superior solubility, preference in this group may be given in turn to the short-chain polyacrylates which have molar masses of from 2 000 to 10 000 g/mol, and particularly preferably from 3 000 to 5 000 g/mol.

Also suitable are copolymeric polycarboxylates, in particular those of acrylic acid with methacrylic acid and of acrylic acid or methacrylic acid with maleic acid. Copolymers which have proven to be particularly suitable are those of acrylic acid with maleic acid which contain from 50 to 90% by weight of acrylic acid and from 50 to 10% by weight of maleic acid. Their relative molecular mass, based on free acids, is generally from 2 000 to 70 000 g/mol, preferably 20 000 to 50 000 g/mol and in particular 30 000 to 40 000 g/mol. The (co)polymeric polycarboxylates may be used either as powders or as aqueous solution. The (co)polymeric polycarboxylates may be from 0.5 to 20% by weight, in particular 1 to 10% by weight of the content of the agent.

To improve the solubility in water, the polymers may also contain allylsulfonic acids such as, for example, allyloxybenzenesulfonic acid and methallylsulfonic acid as monomers.

Particular preference is also given to biodegradable polymers of more than two different monomer units, for example those which contain, as monomers, salts of acrylic acid and of maleic acid, and vinyl alcohol or vinyl alcohol derivatives, or those which contain, as monomers, salts of acrylic acid and of 2-alkylallylsulfonic acid, and sugar derivatives.

Further preferred copolymers are those which preferably have, as monomers, acrolein and acrylic acid/acrylic acid salts or acrolein and vinyl acetate.

Further preferred builder substances which may be mentioned are also polymeric aminodicarboxylic acids, their salts or their precursor substances. Particular preference is given to polyaspartic acids or salts and derivatives thereof.

Further suitable builder substances are polyacetals which can be obtained by reacting dialdehydes with polyolcarboxylic acids having from 5 to 7 carbon atoms and at least 3 hydroxyl groups. Preferred polyacetals are obtained from dialdehydes such as glyoxal, glutaraldehyde, terephthalaldehyde and mixtures thereof and from polyolcarboxylic acids such as gluconic acid and/or glucoheptonic acid.

Further suitable organic builder substances are dextrins, for example oligomers or polymers of carbohydrates, which can be obtained by partial hydrolysis of starches. The hydrolysis can be carried out by customary processes, for example acid-catalyzed or enzyme-catalyzed processes. The hydrolysis products preferably have average molar masses in the range from 400 to 500 000 g/mol. Preference is given here to a polysaccharide having a dextrose equivalent (DE) in the range from 0.5 to 40, in particular from 2 to 30, where DE is a common measure of the reducing action of a polysaccharide compared with dextrose which has a DE of 100. It is possible to use both maltodextrins having a DE between 3 and 20 and dried glucose syrups having a DE between 20 and 37, and also “yellow dextrins” and “white dextrins” with higher molar masses in the range from 2 000 to 30 000 g/mol.

The oxidized derivatives of such dextrins are their reaction products with oxidation agents which are able to oxidize at least one alcohol function of the saccharide ring to the carboxylic acid function. Particularly preferred organic builders for agents of the invention are oxidized starches and derivatives thereof of the applications EP 472042, WO 97/25399 and EP 755944, respectively.

Oxydisuccinates and other derivatives of disuccinates, preferably ethylenediamine disuccinate, are also further suitable cobuilders. Here, ethylenediamine N,N′-disuccinate (EDDS) is preferably used in the form of its sodium or magnesium salts. In this connection, further preference is also given to glycerol disuccinates and glycerol trisuccinates. Suitable use amounts in zeolite-containing, carbonate-containing and/or silicate-containing formulations are between 3 and 15% by weight.

Further organic cobuilders which may be used are, for example, acetylated hydroxycarboxylic acids or salts thereof, which may also be present, where appropriate, in lactone form and which contain at least 4 carbon atoms and at least one hydroxy group and at most two acid groups.

A further class of substance having cobuilder properties is the phosphonates. These are, in particular, hydroxyalkane and aminoalkane phosphonates. Among the hydroxyalkane phosphonates, 1-hydroxyethane 1,1-diphosphonate (HEDP) is of particular importance as a cobuilder. It is preferably used as sodium salt, the disodium salt being neutral and the tetrasodium salt being alkaline (pH 9). Suitable aminoalkane phosphonates are preferably ethylenediaminetetra-methylene phosphonate (EDTMP), diethylenetriamine-pentamethylene phosphonate (DTPMP) and higher homologs thereof. They are preferably used in the form of the neutral sodium salts, for example as the hexasodium salt of EDTMP or as the hepta- and octasodium salt of DTPMP. Here, preference is given to using HEDP as builder from the class of phosphonates. In addition, the aminoalkane phosphonates have a marked heavy metal-binding capacity. Accordingly, particularly if the agents also contain bleaches, it may be preferable to use aminoalkane phosphonates, in particular DTPMP, or mixtures of said phosphonates.

In addition, all compounds which are able to form complexes with alkaline earth metal ions can be used as cobuilders.

The agents of the invention may contain builder substances, where appropriate, in amounts of up to 90% by weight, and preferably contain them in amounts of up to 75% by weight. Detergents of the invention have builder contents of, in particular, from 5% by weight to 50% by weight. In inventive agents for cleaning hard surfaces, in particular for machine cleaning of dishes, the builder substance content is in particular from 5% by weight to 88% by weight, with preferably no water-insoluble builder materials being used in such agents. A preferred embodiment of inventive agents for, in particular, machine cleaning of dishes contains from 20% by weight to 40% by weight water-soluble organic builders, in particular alkali metal citrate, from 5% by weight to 15% by weight alkali metal carbonate and from 20% by weight to 40% by weight alkali metal disilicate.

Solvents which may be used in the liquid to gelatinous compositions of detergents and cleaning agents are, for example, from the group of monohydric or polyhydric alcohols, alkanolamines or glycol ethers, as long as they are miscible with water in the given concentration range. Preferably, the solvents are selected from ethanol, n- or i-propanol, butanols, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl, ethyl or propyl ether, dipropylene glycol monomethyl or monoethyl ether, diisopropylene glycol monomethyl or monoethyl ether, methoxy, ethoxy or butoxy triglycol, 1-butoxyethoxy-2-propanol, 3-methyl-3-methoxybutanol, propylene glycol t-butyl ether, and mixtures of these solvents.

Solvents may be used in the liquid to gelatinous detergents and cleaning agents of the invention in amounts of between 0.1 and 20% by weight, but preferably below 15% by weight, and in particular below 10% by weight.

To adjust the viscosity, one or more thickeners or thickening systems may be added to the composition of the invention. These high molecular weight substances which are also called swell(ing) agents usually soak up the liquids and swell in the process, converting ultimately into viscous true or colloidal solutions.

Suitable thickeners are inorganic or polymeric organic compounds. Inorganic thickeners include, for example, polysilicic acids, clay minerals, such as montmorillonites, zeolites, silicas and bentonites. The organic thickeners are from the groups of natural polymers, modified natural polymers and completely synthetic polymers. Such natural polymers are, for example, agar-agar, carrageen, tragacanth, gum arabic, alginates, pectins, polyoses, guar flour, carob seed flour, starch, dextrins, gelatins and casein. Modified natural substances which are used as thickeners are primarily from the group of modified starches and celluloses. Examples which may be mentioned here are carboxymethylcellulose and other cellulose ethers, hydroxyethylcellulose and hydroxypropylcellulose, and carob flour ether. Completely synthetic thickeners are polymers such as polyacrylic and polymethacrylic compounds, vinyl polymers, polycarboxylic acids, polyethers, polyimines, polyamides and polyurethanes.

The thickeners may be present in an amount up to 5% by weight, preferably from 0.05 to 2% by weight, and particularly preferably from 0.1 to 1.5% by weight, based on the finished composition.

The detergent and cleaning agent of the invention may, where appropriate, comprise, as further customary ingredients, sequestering agents, electrolytes and further excipients such as optical brighteners, graying inhibitors, silver corrosion inhibitors, color transfer inhibitors, foam inhibitors, abrasive substances, dyes and/or fragrances, and microbial active substances and/or UV-absorbing agents.

The textile detergents of the invention may contain, as optical brighteners, derivatives of diaminostilbene-disulfonic acid or alkali metal salts thereof. Suitable are, for example, salts of 4,4′-bis(2-anilino-4-morpholino-1,3,5-triazinyl-6-amino)stilbene-2,2′-disulfonic acid or similarly constructed compounds which carry a diethanolamino group, a methylamino group, an anilino group or a 2-methoxyethylamino group instead of the morpholino group. In addition, brighteners of the substituted diphenylstyryl type may be present, for example the alkali metal salts of 4,4′-bis(2-sulfostyryl)diphenyl, 4,4′-bis(4-chloro-3-sulfostyryl)diphenyl, or 4-(4-chlorostyryl)-4′-(2-sulfostyryl)diphenyl. Mixtures of the above-mentioned optical brighteners may also be used.

Graying inhibitors have the function of keeping the soil detached from the textile fiber in suspension in the liquor. Suitable for this purpose are water-soluble colloids, usually organic in nature, for example starch, glue, gelatin, salts of ethercarboxylic acids or ethersulfonic acids of starch or of cellulose, or salts of acidic sulfuric esters of cellulose or of starch. Water-soluble polyamides containing acidic groups are also suitable for this purpose. Furthermore, starch derivatives other than those mentioned above may be used, for example aldehyde starches. Preference is given to cellulose ethers such as carboxymethyl-cellulose (Na salt), methylcellulose, hydroxyalkylcellulose and mixed ethers such as methylhydroxyethylcellulose, methylhydroxypropylcellulose, methylcarboxymethylcellulose, and mixtures thereof, for example in amounts of from 0.1 to 5% by weight, based on the agents.

In order to protect against silver corrosion, silver corrosion inhibitors may be used in dishwashing cleaning agents of the invention. Such inhibitors are known in the prior art, for example benzotriazoles, iron(III) chloride or CoSO₄. As, for example, European patent EP 0 736 084 B1 discloses, silver corrosion inhibitors which are particularly suitable for being used together with enzymes are manganese, titanium, zirconium, hafnium, vanadium, cobalt, or cerium salts and/or complexes in which the specified metals are present in any of the oxidation stages II, III, IV, V or VI. Examples of such compounds are MnSO₄, V₂O₅, V₂O₄, VO₂, TiOSO₄, K₂TiF₆, K₂ZrF₆, Co(NO₃)₂, Co(NO₃)₃, and mixtures thereof.

Soil-release active ingredients or soil repellents are usually polymers which, when used in a detergent, impart soil-repellent properties to the laundry fiber and/or assist the ability of the other detergent ingredients to detach soil. A comparable effect can also be observed with their use in cleaning agents for hard surfaces.

Soil-release active ingredients which are particularly effective and have been known for a long time are copolyesters having dicarboxylic acid, alkylene glycol and polyalkylene glycol units. Examples thereof are copolymers or mixed polymers of polyethylene terephthalate and polyoxyethylene glycol (DT 16 17 141, and, respectively, DT 22 00 911). German Offenlegungs-schrift DT 22 53 063 discloses acidic agents containing, inter alia, a copolymer of a dibasic carboxylic acid and an alkylene or cycloalkylene polyglycol. German documents DE 28 57 292 and DE 33 24 258 and European patent EP 0 253 567 describe polymers of ethylene terephthalate and polyethylene oxide terephthalate and the use thereof in detergents. European patent EP 066 944 relates to agents containing a copolyester of ethylene glycol, polyethylene glycol, aromatic dicarboxylic acid and sulfonated aromatic dicarboxylic acid in particular molar ratios. European patent EP 0 185 427 discloses methyl or ethyl group end-group-capped polyesters having ethylene and/or propylene terephthalate and polyethylene oxide terephthalate units, and detergents containing such a soil-release polymer. European patent EP 0 241 984 discloses a polyester which contains, in addition to oxyethylene groups and terephthalic acid units also substituted ethylene units and glycerol units. European patent EP 0 241 985 discloses polyesters which contain, in addition to oxyethylene groups and terephthalic acid units, 1,2-propylene, 1,2-butylene and/or 3-methoxy-1,2-propylene groups, and glycerol units and which are end-group-capped with C₁- to C₄-alkyl groups. European patent application EP 0 272 033 discloses polyesters having polypropylene terephthalate and polyoxyethylene terephthalate units, which are at least partially end-group-capped by C₁₋₄-alkyl or acyl radicals. European patent EP 0 274 907 describes sulfoethyl end-group-capped terephthalate-containing soil-release polyesters. According to European patent application EP 0 357 280, sulfonation of unsaturated end groups produces soil-release polyesters having terephthalate, alkylene glycol and poly-C₂₋₄-glycol units. International patent application WO 95/32232 relates to acidic, aromatic polyesters capable of detaching soil. International patent application WO 97/31085 discloses nonpolymeric soil-repellent active ingredients for materials made of cotton, which have a plurality of functional units: a first unit which may be cationic, for example, is able to adsorb to the cotton surface by means of electrostatic interaction, and a second unit which is hydrophobic is responsible for the active ingredient remaining at the water/cotton interface.

The color transfer inhibitors suitable for use in laundry detergents of the invention include, in particular, polyvinylpyrrolidones, polyvinylimidazoles, polymeric N-oxides such as poly(vinylpyridine N-oxide) and copolymers of vinylpyrrolidone with vinylimidazole.

For use in machine cleaning processes, it may be of advantage to add foam inhibitors to the agents. Examples of suitable foam inhibitors are soaps of natural or synthetic origin having a high proportion of C₁₈-C₂₄ fatty acids. Examples of suitable nonsurfactant-type foam inhibitors are organopolysiloxanes and their mixtures with microfine, optionally silanized silica and also paraffins, waxes, microcrystalline waxes, and mixtures thereof with silanized silica or bis-stearyl-ethylenediamide. With advantages, use is also made of mixtures of different foam inhibitors, for example mixtures of silicones, paraffins or waxes. The foam inhibitors, in particular those containing silicone and/or paraffin, are preferably bound to a granular, water-soluble or dispersible support substance. Particular preference is given here to mixtures of paraffins and bis-stearylethylenediamides.

A cleaning agent of the invention for hard surfaces may, in addition, contain ingredients with abrasive action, in particular from the group comprising quartz flours, wood flours, polymer flours, chalks and glass microbeads, and mixtures thereof. Abrasives are present in the cleaning agents of the invention preferably at not more than 20% by weight, in particular from 5% by weight to 15% by weight.

Dyes and fragrances are added to detergents and cleaning agents in order to improve the esthetic appeal of the products and to provide the consumer, in addition to washing and cleaning performance, with a visually and sensorially “typical and unmistakable” product. As perfume oils and/or fragrances it is possible to use individual odorant compounds, for example the synthetic products of the ester, ether, aldehyde, ketone, alcohol and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenyl glycinate, allylcyclohexyl propionate, styrallyl propionate and benzyl salicylate. The ethers include, for example, benzyl ethyl ether; the aldehydes include, for example, the linear alkanals having 8-18 carbon atoms, citral, citronellal, citronellyloxyacetaldehyde, cyclamenaldehyde, hydroxycitronellal, lilial and bourgeonal; the ketones include, for example, the ionones, α-isomethylionone and methyl cedryl ketone; the alcohols include anethol, citronellol, eugenol, geraniol, linalool, phenylethyl alcohol, and terpineol; the hydrocarbons include primarily the terpenes such as limonene and pinene. Preference, however, is given to the use of mixtures of different odorants which together produce an appealing fragrance note. Such perfume oils may also contain natural orodant mixtures, as obtainable from plant sources, for example pine oil, citrus oil, jasmine oil, patchouli oil, rose oil or ylang-ylang oil. Likewise suitable are muscatel, sage oil, camomile oil, clove oil, balm oil, mint oil, cinnamon leaf oil, lime blossom oil, juniper berry oil, vetiver oil, olibanum oil, galbanum oil and labdanum oil, and also orange blossom oil, neroli oil, orangepeel oil and sandalwood oil. The dye content of detergents and cleaning agents is usually less than 0.01% by weight, while fragrances may make up up to 2% by weight of the overall formulation.

The fragrances may be incorporated directly into the detergents and cleaning agents; however, it may also be advantageous to apply the fragrances to carriers which intensify the adhesion of the perfume to the material to be cleaned and, by means of slower fragrance release, ensure long-lasting fragrance, in particular of treated textiles. Materials which have become established as such carriers are, for example, cyclodextrins, it being possible, in addition, for the cyclodextrin-perfume complexes to be additionally coated with further auxiliaries. Another preferred carrier for fragances is the described zeolite X which can also absorb fragrances instead of or in a mixture with surfactants. Preference is therefore given to detergents and cleaning agents which contain the described zeolite X and fragrances which, preferably, are at least partially absorbed on the zeolite.

Preferred dyes whose selection is by no means difficult for the skilled worker have high storage stability and insensitivity to the other ingredients of the agents and to light, and also have no pronounced affinity for textile fibers, so as not to stain them.

To control microorganisms, detergents or cleaning agents may contain antimicrobial active ingredients. Depending on antimicrobial spectrum and mechanism of action, a distinction is made here between bacteriostatics and bactericides, fungistatics and fungicides, etc. Examples of important substances from these groups are benzalkonium chlorides, alkylaryl sulfonates, halogen phenols and phenol mercury acetate. The terms antimicrobial action and antimicrobial active ingredient have, within the teaching of the invention, the meaning common in the art, which is described, for example, by K. H. Wallhäuβer in “Praxis der Sterilisation, Desinfektion—Konservierung: Keimidentifizierung—Betriebshygiene” (5th Edition,—Stuttgart; New York: Thieme, 1995), it being possible to use all of the substances having antimicrobial action described there. Suitable antimicrobial active ingredients are preferably selected from the groups of alcohols, amines, aldehydes, antimicrobial acids or their salts, carboxylic esters, acid amides, phenols, phenol derivatives, diphenyls, diphenylalkanes, urea derivatives, oxygen acetals, nitrogen acetals and also oxygen and nitrogen formals, benzamidines, isothioazolines, phthalimide derivatives, pyridine derivatives, antimicrobial surfactant compounds, guanidines, antimicrobial amphoteric compounds, quinolines, 1,2-dibromo-2,4-dicyanobutane, iodo-2-propylbutyl carbamate, iodine, iodophors, peroxo compounds, halogen compounds, and any mixtures of the above.

The antimicrobial active ingredient may be selected from ethanol, n-propanol, isopropanol, 1,3-butanediol, phenoxyethanol, 1,2-propylene glycol, glycerol, undecylenic acid, benzoic acid, salicylic acid, dihydracetic acid, o-phenylphenol, N-methylmorpholino-acetonitrile (MMA), 2-benzyl-4-chlorophenol, 2,2′-methylenebis(6-bromo-4-chlorophenol), 4,4′-dichloro-2′-hydroxydiphenyl ether (dichlosan), 2,4,4′-trichloro-2′-hydroxydiphenyl ether (trichlosan), chlorohexidine, N-(4-chlorophenyl)-N-(3,4-dichlorophenyl)urea, N,N′-(1,10-decanediyldi-1-pyridinyl-4-ylidene)-bis(1-octanamine) dihydrochloride, N,N′-bis(4-chlorophenyl)-3,12-diimino-2,4,11,13-tetra-azatetradecanediimidamide, glucoprotamines, antimicrobial surface-active quaternary compounds, guanidines including the bi- and polyguanidines, such as, for example, 1,6-bis(2-ethylhexylbiguanidohexane) dihydrochloride, 1,6-di-(N₁,Nₗ′-phenyldiguanido-N₅,N₅′)hexane tetrahydrochloride, 1,6-di-(N₁,N₁′-phenyl-N₁,N₁-methyldiguanido-N₅,N₅′)hexane dihydrochloride, 1,6-di-(N₁,N₁′-o-chlorophenyldiguanido-N₅,N₅′)hexane dihydrochloride, 1,6-di-(N₁,N₁′-2,6-dichlorophenyl-diguanido-N₅,N₅′)hexane dihydrochloride, 1,6-di-[N₁,N₁′-beta-(p-methoxyphenyl)diguanido-N₅,N₅′]hexane dihydrochloride, 1,6-di-(N₁,N₁′-alpha-methyl-beta-phenyldiguanido-N₅,N₅′)hexane dihydrochloride, 1,6-di-(N₁,N₁′-p-nitrophenyldiguanido-N₅,N₅′)hexane dihydrochloride, omega:omega-di-(N₁,Nₗ′-phenyldiguanido-N₅,N₅′)-di-n-propyl ether dihydrochloride, omega:omega′-di-(N₁,N₁′-p-chlorophenyldiguanido-N₅,N₅′)-di-n-propyl ether tetrahydrochloride, 1,6-di-(N₁,N₁′-2,4-dichlorophenyldiguanido-N₅,N₅′)hexane tetrahydrochloride, 1,6-di-(N₁,N₁′-p-methylphenyldiguanido-N₅,N₅′)hexane dihydrochloride, 1,6-di-(N₁,N₁′-2,4,5-trichlorophenyldiguanido-N₅,N₅′)hexane tetrahydrochloride, 1,6-di-[N₁,N₁′-alpha-(p-chlorophenyl)ethyldiguanido-N₅,N₅′]-hexane dihydrochloride, omega: omega-di-(N₁,N₁′-p-chlorophenyldiguanido-N₅,N₅′)m-xylene dihydrochloride, 1,12-di-(N₁,N₁′-p-chlorophenyldiguanido-N₅,N₅′)dodecane dihydrochloride, 1,10-di-(N₁,N₁′-phenyldiguanido-N₅,N₅′)decane tetrahydrochloride, 1,12-di-(N₁,N₁′-phenyldiguanido-N5,N₅′)dodecane tetrahydrochloride, 1,6-di-(N₁,N₁′-o-chlorophenyldiguanido-N₅,N₅′)hexane dihydrochloride, 1,6-di-(N₁,N₁′-o-chlorophenyldiguanido-N₅,N₅′)hexane tetrahydrochloride, ethylene-bis(1-tolylbiguanide), ethylene-bis(p-tolylbiguanide), ethylene-bis(3,5-dimethylphenylbiguanide), ethylene-bis(p-tert-amylphenylbiguanide), ethylene-bis(nonyl-phenylbiguanide), ethylene-bis(phenylbiguanide), ethylene-bis(N-butylphenylbiguanide), ethylene-bis(2,5-diethoxyphenylbiguanide), ethylene-bis(2,4-dimethylphenylbiguanide), ethylene-bis(o-diphenylbiguanide), ethylene-bis(mixed amyl naphthylbiguanide), N-butylethylene-bis(phenylbiguanide), trimethylenebis(o-tolylbiguanide), N-butyl-trimethyl-bis(phenylbiguanide) and the corresponding salts such as acetates, gluconates, hydrochlorides, hydrobromides, citrates, bisulfites, fluorides, polymaleates, N-cocoalkyl sarcosinates, phosphites, hypophosphites, perfluorooctanoates, silicates, sorbates, salicylates, maleates, tartrates, fumarates, ethylenediaminetetraacetates, iminodiacetates, cinnamates, thiocyanates, arginates, pyromellitates, tetracarboxybutyrates, benzoates, glutarates, monofluorophosphates, perfluoropropionates, and any mixtures thereof. Also suitable are halogenated xylene and cresol derivatives, such as p-chlorometacresol or p-chlorometaxylene, and natural antimicrobial active ingredients of plant origin (for example from spices or herbs), animal origin and microbial origin. Preference may be given to using antimicrobial surface-active quaternary compounds, a natural antimicrobial active ingredient of plant origin and/or a natural antimicrobial active ingredient of animal origin, most preferably at least one natural antimicrobial active ingredient of plant origin from the group comprising caffeine, theobromine and theophylline and essential oils such as eugenol, thymol and geraniol, and/or at least one natural antimicrobial active ingredient of animal origin from the group comprising enzymes such as milk protein, lysozyme and lactoperoxidase, and/or at least one antimicrobial surface-active quaternary compound having an ammonium, sulfonium, phosphonium, iodonium or arsonium group, peroxo compounds and chlorine compounds. It is also possible to use substances of microbial origin, the “bacteriocines”.

The quaternary ammonium compounds (QACs) which are suitable as antimicrobial active ingredients have the general formula (R¹) (R²) (R³) (R⁴) N⁺ X⁻ where R¹ to R⁴ are identical or different C₁-C₂₂-alkyl radicals, C₇-C₂₈-aralkyl radicals or heterocyclic radicals, where two, or in the case of an aromatic incorporation as in pyridine, even three radicals, together with the nitrogen atom, form the heterocycle, for example a pyridinium or imidazolinium compound, and X⁻ are halide ions, sulfate ions, hydroxide ions or similar anions. For optimal antimicrobial action, at least one of the radicals preferably has a chain length of from 8 to 18, in particular 12 to 16, carbon atoms.

QACs can be prepared by reacting tertiary amines with alkylating agents such as, for example, methyl chloride, benzyl chloride, dimethyl sulfate, dodecyl bromide, or else ethylene oxide. The alkylation of tertiary amines having one long alkyl radical and two methyl groups proceeds particularly readily, and the quaternization of tertiary amines having two long radicals and one methyl group can also be carried out with the aid of methyl chloride under mild conditions. Amines which have three long alkyl radicals or hydroxy-substituted alkyl radicals have low reactivity and are preferably quaternized using dimethyl sulfate.

Examples of suitable QACs are benzalkonium chloride (N-alkyl-N,N-dimethylbenzylammonium chloride, CAS No. 8001-54-5), benzalkone B (m,p-dichlorobenzyldimethyl-C12-alkylammonium chloride, CAS No. 58390-78-6), benzoxonium chloride (benzyldodecyl-bis(2-hydroxyethyl)ammonium chloride), cetrimonium bromide (N-hexadecyl-N,N-trimethylammonium bromide, CAS No. 57-09-0), benzetonium chloride (N,N-dimethyl-N-[2-[2-[p-(1,1,3,3-tetramethylbutyl)phenoxy]ethoxy]ethyl]-benzylammonium chloride, CAS No. 121-54-0), dialkyldimethylammonium chlorides such as di-n-decyldimethylammonium chloride (CAS No. 7173-51-5-5), didecyldimethylammonium bromide (CAS No. 2390-68-3), dioctyldimethylammonium chloride, 1-cetylpyridinium chloride (CAS No. 123-03-5) and thiazoline iodide (CAS No. 15764-48-1), and mixtures thereof. Particularly preferred QACs are the benzalkonium chlorides having C₈-C₁₈-alkyl radials, in particular C₁₂-C₁₄-alkyl-benzyldimethylammonium chloride.

Benzalkonium halides and/or substituted benzalkonium halides are commercially available, for example, as Barquat® ex Lonza, Marquat® ex Mason, Variquat® ex Witco/Sherex and Hyamine® ex Lonza, and Bardac® ex Lonza. Further commercially available antimicrobial active ingredients are N-(3-chloroallyl)hexaminium chloride such as Dowicide® and Dowicil® ex Dow, benzethonium chloride such as Hyamine® 1622 ex Rohm & Haas, methylbenzethonium chloride such as Hyamine® 10X ex Rohm & Haas, cetylpyridinium chloride such as cepacol chloride ex Merrell Labs.

The antimicrobial active ingredients are used in amounts of from 0.0001% by weight to 1% by weight, preferably from 0.001% by weight to 0.8% by weight, particularly preferably from 0.005% by weight to 0.3% by weight, and in particular from 0.01 to 0.2% by weight.

The agents may contain UV absorbers which attach to the treated textiles and improve the light stability of the fibers and/or the light stability of other formulation constituents. UV absorbers mean organic substances (light protection filters) which are able to absorb ultraviolet radiation and to emit the absorbed energy again in the form of radiation of longer wavelength, for example heat.

Compounds which have these desired properties are, for example, the compounds which are active via radiationless deactivation and derivatives of benzophenone having substituents in position(s) 2 and/or 4. Furthermore, also suitable are substituted benzotriazoles, acrylates which are phenyl-substituted in position 3 (cinnamic acid derivatives, with or without cyano groups in position 2), salicylates, organic Ni complexes and natural substances such as umbelliferone and the endogenous urocanic acid. Of particular importance are biphenyl and especially stilbene derivatives, as described, for example, in EP 0728749 A and commercially available as Tinosorb® FD or Tinosorb® FR ex Ciba. UV-B absorbers which may be mentioned are: 3-benzylidenecamphor or 3-benzylidenenorcamphor and derivatives thereof, for example 3-(4-methylbenzylidene)camphor, as described in EP 0693471 B1; 4-aminobenzoic acid derivatives, preferably 2-ethylhexyl 4-(dimethylamino)benzoate, 2-octyl 4-(dimethylamino)benzoate and amyl 4-(dimethylamino)benzoate; esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (octocrylenes); esters of salicylic acid, preferably 2-ethylhexyl salicylate, 4-isopropylbenzyl salicylate, homomenthyl salicylate; derivatives of benzophenone, preferably 2-hydroxy-4-methoxybenzophenone, 2-hydroxy-4-methoxy-4′-methylbenzophenone, 2,2′-dihydroxy-4-methoxybenzo-phenone; esters of benzalmalonic acid, preferably di-2-ethylhexyl 4-methoxybenzmalonate; triazine derivatives such as, for example, 2,4,6-trianilino-(p-carbo-2′-ethyl-l′-hexyloxy)-1,3,5-triazine and octyltriazone, as described in EP 0818450 A1, or dioctylbutamidotriazones (Uvasorb® HEB); propane-1,3-diones such as, for example, 1-(4-tert-butylphenyl)-3-(4′-methoxyphenyl)propane-1,3-dione; ketotricyclo(5.2.1.0)decane derivatives, as described in EP 0694521 B1. Further suitable are 2-phenylbenzimidazole-5-sulfonic acid and its alkali metal, alkaline earth metal, ammonium, alkylammonium, alkanolammonium and glucammonium salts; sulfonic acid derivatives of benzophenones, preferably 2-hydroxy-4-methoxybenzophenone-5-sulfonic acid and its salts; sulfonic acid derivatives of 3-benzylidenecamphor, such as, for example, 4-(2-oxo-3-bornylidenemethyl)benzenesulfonic acid and 2-methyl-5-(2-oxo-3-bornylidene)sulfonic acid and salts thereof.

Suitable typical UV-A filters are, in particular, derivatives of benzoylmethane, such as, for example, 1-(4′-tert-butylphenyl)-3-(4′-methoxyphenyl)propane-1,3-dione, 4-tert-butyl-4′-methoxydibenzoylmethane (Parsol 1789), 1-phenyl-3-(4′-isopropylphenyl)propane-1,3-dione, and enamine compounds, as described in DE 19712033 A1 (BASF). The UV-A and UV-B filters may of course also be used in mixtures. In addition to said soluble substances, insoluble light protection pigments, namely finely dispersed, preferably nanoized, metal oxides or salts, are also suitable for this purpose. Examples of suitable metal oxides are, in particular, zinc oxide and titanium dioxide and also oxides of iron, zirconium, silicon, manganese, aluminum and cerium, and mixtures thereof. Salts which may be used are silicates (talc), barium sulfate or zinc stearate. The oxides and salts are already used in the form of the pigments for skin-care and skin-protective emulsions and decorative cosmetics. The particles here should have an average diameter of less than 100 nm, preferably between 5 and 50 nm, and in particular between 15 and 30 nm. They can have a spherical shape, but it is also possible to use particles which have an ellipsoidal shape or a shape deviating in some other way from the spherical form. The pigments may also be surface-treated, i.e. hydrophilicized or hydrophobicized. Typical examples are coated titanium dioxides such as, for example, titanium dioxide T 805 (Degussa) or Eusolex® T2000 (Merck); suitable hydrophobic coating agents are here preferably silicones and, particularly preferably, trialkoxyoctylsilanes or simethicones. Preference is given to using micronized zinc oxide. Further suitable UV light protection filters can be found in the review by P. Finkel in SÖFW-Journal 122 (1996), p. 543.

The UV absorbers are usually used in amounts of from 0.01% by weight to 5% by weight, preferably from 0.03% by weight to 1% by weight.

The ingredients usual for detergents and cleaning agents usually also include detersive and, respectively, cleaning-active enzymes. At the same time, detergents or cleaning agents which are additionally characterized by further enzymes in addition to a protein of the invention are preferred embodiments of the present invention. Examples of these include other proteases but also oxidoreductases, cutinases, esterases and/or hemicellulases, and particularly preferably lipases, amylases, cellulases and/or β-glucanases.

Enzymes such as proteases, amylases, lipases or cellulases have been used for decades as active components in detergents and cleaning agents. Their particular contribution to the washing and, respectively, cleaning performance of the agent in question is, in the case of protease, the ability to break down proteinaceous soilings, in the case of amylase, the breaking-down of starch-containing soilings and, in the case of lipase, fat-cleaving activity. Cellulases are preferably used in detergents, in particular due to their contribution to the secondary washing performance of a detergent and due to their fiber action on textiles, in addition to their soil-removing, i.e. primary washing and cleaning performance. The particular hydrolytic products are attacked, dissolved, emulsified or suspended by the other detergent or cleaning agent components or are, due to their greater solubility, washed away with the wash liquor, resulting in synergistic effects between the enzymes and the other components.

Proteases can exert an effect on natural fibers, in particular on wool or silk, which is comparable to the contribution by cellulase to the secondary washing performance of an agent. Due to their action on the surface structure of such fabrics, they can exert a smoothing influence on the material and thereby counteract felting.

Other enzymes extend the cleaning performance of appropriate agents by their in each case specific enzyme performance. Examples of this include β-glucanases (WO 99/06515 and WO 99/06516), laccases (WO 00/39306) or pectin-dissolving enzymes (WO 00/42145) which are used, in particular, in special detergents.

Enzymes suitable for use in agents of the invention are primarily those from microorganisms such as bacteria or fungi. They are obtained from suitable microorganisms in a manner known per se by means of fermentation processes which are described, for example, in German Laid-Open Specifications DE 1940488, and DE 2121397, the U.S. Pat. Nos. 3,623,957, 4264738, European patent application EP 006638 and international patent application WO 91/02792.

Particularly during storage, a protein of the invention and/or other proteins present may be protected by stabilizers from, for example, denaturation, decay or inactivation, for example by physical influences, oxidation or proteolytic cleavage.

One group of stabilizers are reversible protease inhibitors which dissociate off when diluting the agent in the wash liquor. Benzamidine hydrochloride and leupeptin are established for this purpose. Frequently, borax, boric acids, boronic acids or salts or esters thereof are used, including especially derivatives with aromatic groups, for example, according to WO 95/12655, ortho-substituted, according to WO 92/19707, meta-substituted and, according to U.S. Pat. No. 5,972,873, para-substituted phenylboronic acids, or salts or esters thereof. The applications WO 98/13460 and EP 583534 disclose peptide aldehydes, i.e. oligopeptides with reduced C terminus, that is those of 2-50 monomers, for the reversible inhibition of detergent and cleaning agent proteases. The peptidic reversible protease inhibitors include, inter alia, ovomucoid (WO 93/00418). For example, the application WO 00/01826 discloses specific reversible peptide inhibitors of the protease Subtilisin for use in protease-containing agents, and WO 00/01831 discloses corresponding fusion proteins of protease and inhibitor.

Further enzyme stabilizers are amino alcohols such as mono-, di-, triethanol- and -propanolamine and mixtures thereof, aliphatic carboxylic acids up to C₁₂, as disclosed, for example, by the applications EP 0378261 and WO 97/05227, such as succinic acid, other dicarboxylic acids or salts of said acids. The application DE 19650537 discloses end group-capped fatty amide alkoxylates for this purpose. As disclosed in WO 97/18287, particular organic acids used as builders are capable of additionally stabilizing a contained enzyme.

Lower aliphatic alcohols, but especially polyols such as, for example, glycerol, ethylene glycol, propylene glycol or sorbitol, are other frequently used enzyme stabilizers. Calcium salts are also used, such as, for example, calcium acetate or the calcium formate disclosed for this purpose in EP 0028865, and magnesium salts, for example according to the European Application EP 0378262.

Polyamide oligomers (WO 99/43780) or polymeric compounds such as lignin (WO 97/00932), water-soluble vinyl copolymers (EP 828 762) or, as disclosed in EP 702 712, cellulose ethers, acryl polymers and/or polyamides stabilize the enzyme preparation inter alia against physical influences or pH fluctuations. Polyamine N-oxide-containing polymers (EP 587550 and EP 581751) simultaneously act as enzyme stabilizers and as color transfer inhibitors. Other polymeric stabilizers are the linear C₈-C₁₈ polyoxyalkylenes disclosed, in addition to other components, in WO 97/05227. As in the applications WO 97/43377 and WO 98/45396, alkylpolyglycosides could stabilize the enzymic components of the agent of the invention and even increase their performance. Crosslinked N-containing compounds, as disclosed in WO 98/17764, fulfill a double function as soil release agents and as enzyme stabilizers. Hydrophobic, nonionic polymer acts in a mixture together with other stabilizers, according to the application WO 97/32958, in a stabilizing manner on a cellulase so that those or similar components may also be suitable for the enzyme essential to the invention.

As disclosed inter alia in EP 780466, reducing agents and antioxidants increase the stability of the enzymes against oxidative decay. Sulfur-containing reducing agents are disclosed, for example, in EP 0080748 and EP 0080223. Other examples are sodium sulfite (EP 533239) and reducing sugars (EP 656058).

Frequently used are also combinations of stabilizers, for example of polyols, boric acid and/or borax in the application WO 96/31589, the combination of boric acid or borate, reducing salts and succinic acid or other dicarboxylic acids in the application EP 126505 or the combination of boric acid or borate with polyols or polyamino compounds and with reducing salts, as disclosed in the application EP 080223. According to WO 98/13462, the action of peptide-aldehyde stabilizers is increased by combination with boric acid and/or boric acid derivatives and polyols and, according to WO 98/13459, still further increased by the additional use of calcium ions.

Agents containing stabilized enzyme activities are preferred embodiments of the present invention. Particular preference is given to those containing enzymes stabilized in a plurality of the manners indicated.

Since agents of the invention can be provided in any conceivable form, enzymes or proteins of the invention in any formulations appropriate for addition to the particular agents are respective embodiments of the present invention. Examples thereof include liquid formulations, solid granules or capsules.

The encapsulated form is a way of protecting the enzymes or other ingredients against other components such as, for example, bleaches, or of making possible a controlled release. Depending on their size, said capsules are divided into milli-, micro- and nanocapsules, microcapsules being particularly preferred for enzymes. Such capsules are disclosed, for example, in the patent applications WO 97/24177 and DE 199 18 267. A possible encapsulation method is to encapsulate the proteins, starting from a mixture of the protein solution with a solution or suspension of starch or a starch derivative, in this substance. German application DE 199 56 382 entitled Verfahren zur Herstellung von mikroverkapselten Enzymen [Method for preparing microencapsulated enzymes] describes such an encapsulation method.

In the case of solid agents, the proteins may be used, for example, in dried, granulated and/or encapsulated form. They may be added separately, i.e. as a separate phase, or together with other components in the same phase, with or without compaction. If microencapsulated enzymes have to be processed in solid form, it is possible to remove the water from the aqueous solutions resulting from the work-up by using methods known in the prior art, such as spray drying, removing by centrifugation or resolubilizing. The particles obtained in this way are usually between 50 and 200 μgm in size.

It is possible to add to liquid, gel-like or paste-like agents of the invention the enzymes and also the protein important to the invention, starting from protein recovery carried out according to the prior art, and preparation in a concentrated aqueous or nonaqueous solution, suspension or emulsion, but also in gel form or encapsulated or as dried powder. Such detergents or cleaning agents of the invention are usually prepared by simply mixing the ingredients which may be introduced as solids or as solution into an automated mixer.

Apart from the primary washing performance, the proteases present in detergents may further fulfill the function of activating, or, after an appropriate period of action, inactivating other enzymic components by proteolytic cleavage, as disclosed, for example, in the applications WO 94/29426 and EP 747 471. Comparable regulatory functions are also possible via the enzyme of the invention. Another embodiment of the present invention relates to those agents containing capsules of protease-sensitive material, which capsules are hydrolyzed, for example, by proteins of the invention at the intended time and release their contents. A comparable effect may also be achieved in other multi-phase agents.

Agents for the treatment of textile raw materials or for textile care, which are characterized in that they contain a proteolytic enzyme of the invention, either alone or in addition to other ingredients, are a separate subject matter of the invention, since natural fibers in particular, such as wool or silk, for example, are distinguished by a characteristic, microscopic surface structure. Said surface structure can, in the long term, result in undesired effects such as, for example, felting, as discussed by way of example for wool in the article by R. Breier in Melliand Textilberichte from 4.1.2000 (p. 263). In order to avoid such effects, the natural raw materials are treated with agents of the invention which contribute, for example, to smoothing the flaked surface structure based on protein structures and thereby counteract felting. Agents of this kind for fibers or textiles containing natural components and, very particularly, containing wool or silk are a particularly preferred embodiment.

In one embodiment of the present invention, the agent containing a protease of the invention is designed in such a way that it can be used regularly as a care agent, for example by adding it to the washing process, applying it after washing or independently of the washing. The desired effect is to obtain a smooth surface structure of the textile and/or to prevent and/or reduce damage to the fabric.

Methods for machine cleaning of textiles or of hard surfaces, which methods are characterized in that a proteolytic enzyme of the invention becomes active in at least one of the method steps, are a separate subject matter of the invention.

Methods for machine cleaning of textiles are generally distinguished by several method steps comprising applying various cleaning-active substances to the material to be cleaned and, after the time of action, washing them off, or by the material to be cleaned being treated in any other way with a cleaning agent or a solution of said agent. The same applies to methods for machine cleaning of any other materials as textiles which are classified under the term hard surfaces. It is possible to add proteins of the invention to at least one of the method steps of such methods, which methods then become embodiments of the present invention.

Preference is given to methods in which an enzyme of the invention is used in an amount of from 40 μg to 4 g and, more preferably, from 50 μg to 3 g, from 100 μg to 2 g, from 200 μg to 1 g and, particularly preferably, from 400 μg to 400 mg per application.

Since the enzyme of the invention already by nature possesses a protein-dissolving activity and also exhibits said activity in media which otherwise have no cleaning power, such as, for example, in mere buffer, an individual partial step of such a method for machine cleaning of textiles may consist of applying, if desired in addition to stabilizing compounds, salts or buffer substances, the enzyme of the invention as single cleaning-active component. This is a particularly preferred embodiment of the present invention.

Methods for the treatment of textile raw materials or textile care, which methods are characterized in that a proteolytic enzyme of the invention becomes active in at least one of the method steps, are a separate subject matter of the invention. They may be, for example, methods in which materials are prepared for use in textiles, for example for anti-felt finishing, or, for example, methods which add a care component to the cleaning of worn textiles. Due to the above-described action of proteases on particular fabrics, particular embodiments comprise textile raw materials or textiles containing natural components, in particular containing wool or silk.

The use of a proteolytic enzyme of the invention for cleaning textiles or hard surfaces is a separate subject matter of the invention, since enzymes of the invention may be used, in particular according to the above-described methods, in order to remove proteinaceous soilings from textiles or from hard surfaces. The use outside a machine-based method, for example in manual laundry or manual removal of stains from textiles or from hard surfaces are preferred embodiments.

Preference is given to using an enzyme of the invention in an amount of from 40 μg to 4 g and, more preferably, from 50 μg to 3 g, from 100 μg to 2 g, from 200 μg to 1 g and, particularly preferably, from 400 μg to 400 mg per application.

The use of a proteolytic enzyme of the invention for activating or deactivating ingredients of detergents or cleaning agents is a separate subject matter of the invention, since protein components of detergents or cleaning agents, as is known, can be inactivated by the action of a protease. The present invention relates to specifically using this otherwise rather undesired effect. It is likewise possible that proteolysis only activates another component, for example if said component is a hybrid protein of the actual enzyme and the corresponding inhibitor, as disclosed, for example, in the application WO 00/01831. Another example of a regulation of this kind is one in which an active component, in order to protect or control its activity, has been encapsulated in a material susceptible to proteolytic attack. Proteins of the invention can thus be used for inactivation reactions, activation reactions or release reactions.

The use of a proteolytic enzyme of the invention for biochemical or molecular-biological analysis, in particular within the framework of an enzymic analytical method, is a separate subject matter of the invention. According to the invention and according to Rbmpp, “Lexikon Chemie” (Version 2.0, Stuttgart/New York: Georg Thieme Verlag, 1999), enzymic analysis means any biochemical analysis which uses specific enzymes or substrates in order to determine, on the one hand, the identity or concentration of substrates or, on the other hand, the identity or activity of enzymes. Areas of application are any areas of work related to biochemistry. A preferred embodiment of this subject matter of the invention is the use for determining the terminal groups in a sequence analysis.

The use of a proteolytic enzyme of the invention for the preparation, purification or synthesis of natural substances or biological valuable substances is a separate subject matter of the invention. Thus, it may be necessary, for example, within the course of purifying natural substances or biological valuable substances to remove from said substances protein contaminations, examples of which are low molecular weight compounds, any cellular constituents or storage substances or proteins. This may be carried out both on the laboratory scale and the industrial scale, for example after biotechnological production of a valuable substance.

A proteolytic enzyme of the invention is used for the synthesis of proteins or other low molecular weight chemical compounds by reversing the reaction which they catalyze by nature, for example when it is intended to link protein fragments to one another or to bind amino acids to a compound which is not predominantly composed of protein. Possible uses of this kind are introduced, for example, in the application EP 380362.

The use of a proteolytic enzyme of the invention for the treatment of natural raw materials is a separate subject matter of the invention, if it is intended to remove protein contaminations from said raw materials, which mean primarily raw materials which are obtained non-microbiologically, for example those from agriculture.

A preferred embodiment is the use for the treatment of surfaces, and very particularly in a method for the treatment of the economically important raw material leather. Thus, water-soluble proteins are removed from the skin material with the aid of proteolytic enzymes during the tanning process, in particular in the step of alkaline steep (Römpp, “Lexikon Chemie”, Version 2.0, Stuttgart/New York: Georg Thieme Verlag, 1999). Proteases of the invention are suitable for this, in particular under alkaline conditions and in the presence of denaturing agents.

The use of a proteolytic enzyme of the invention for the obtainment or treatment of raw materials or intermediates in the manufacture of textiles is a separate subject matter of the invention. An example thereof is the work-up of cotton from which capsule components need to be removed in a process referred to as sizing; another example is the treatment of wool; the processing of raw silk is also similar. Enzymic methods are superior to comparable chemical methods, in particular with respect to their environmental compatibility.

In a preferred embodiment, proteins of the invention are used for removing protective layers from textiles, in particular from intermediate products or valuable substances, or smoothing their surface, before further treatment in a subsequent processing step.

In a separate subject matter of the invention, proteins of the invention are used for the treatment of textile raw materials or for textile care, in particular for the treatment of surfaces of wool or silk or of wool- or silk-containing mixed textiles. This applies both to the preparation for such textiles and to the care during usage, for example in connection with the cleaning of textiles (see above).

The use of a proteolytic enzyme of the invention for the treatment of photographic films, in particular for removing gelatin-containing or similar protective layers, is a separate subject matter of the invention, since films such as, for example, X-ray films, are coated with such protective layers, in particular those made of silver salt-containing gelatin emulsions, which films need to be removed from the support material after exposure. For this, proteases of the invention may be used, in particular under alkaline or slightly denaturing reaction conditions.

The use of a proteolytic enzyme of the invention for preparing food or animal feed is a separate subject matter of the invention. Thus proteases have been used for the preparation of food from time immemorial. An example of this is the use of rennet for the maturing process of cheese or other milk products. A protein of the invention may be added to or used to completely carry out such processes. Carbohydrate-rich food or food raw materials for non-nutritional purposes, such as, for example, flour or dextrin, may also be treated with appropriate proteases in order to remove accompanying proteins from them. A protease of the invention is suitable for those applications, too, in particular if they are intended to be carried out under alkaline or slightly denaturing conditions.

This applies accordingly for the preparation of animal feed. In addition to a complete removal of proteins, it may also be of interest here to treat the proteinaceous starting substances or substance mixtures with proteases only for a short time in order to render them more readily digestible for domestic animals.

Cosmetic agents containing a proteolytic enzyme of the invention or cosmetic methods incorporating a proteolytic enzyme of the invention or the use of a proteolytic enzyme of the invention for cosmetic purposes, in particular within the framework of corresponding methods or in corresponding agents, are a separate subject matter of the invention.

Since proteases also play a crucial part in the desquamation of human skin (T. Egelrud et al., Acta Derm. Venerol., volume 71 (1991), pp. 471-747), accordingly, proteases are also used as bioactive components in skincare products in order to support degradation of the desmosome structures increasingly present in dry skin, for example according to the applications WO 95/07688 and WO 99/18219. WO 97/07770, for example, describes the use of subtilisin proteases, in particular of the B. lentus alkaline protease variants described above, for cosmetic purposes. Proteases of the invention, in particular those whose activity is controlled, for example after mutagenesis or due to addition of appropriate substances interacting with them, are also suitable as active components in skin- or hair-cleaning compositions or care compositions. Particular preference is given to those preparations of said enzymes, which, as described above, are stabilized, for example by coupling to macromolecular supports (compare U.S. Pat. No. 5,230,891), and/or are derivatized by point mutations at highly allergenic positions so that their compatibility with human skin is increased.

Accordingly, the use of proteolytic enzymes of this kind for cosmetic purposes, in particular in appropriate agents such as, for example, shampoos, soaps or washing lotions or in care compositions provided, for example, in the form of creams, is also included in this subject matter of the invention. The use in a peeling medicament is also included in this claim.

### EXAMPLES

### Example 1

Generation of the Protease of the Invention

All molecular-biological working steps follow standard methods as indicated, for example, in the manual by Fritsch, Sambrook and Maniatis “Molecular cloning: a laboratory manual”, Cold Spring Harbour Laboratory Press, New York, 1989, or in international patent application WO 92/21760.

Construction of the Mutagenesis Vector

The mutagenesis was carried out starting from the protease variant B. lentus alkaline protease M131. This variant is described in WO 92/21760 and the strain according to this application, which produces it, has been deposited with the American Type Culture Collection, Rockville, Md., USA under the name Bacillus licheniformis ATCC 68614. This strain contains the gene on plasmid pCB56M131 which replicates in Bacillus in an expression cassette comprising the promoter, the ribosomal binding site and the ATG start codon and the 22 amino-terminal amino acids of the alkaline protease from Bacillus licheniformis ATCC 53926 which are fused to the prepro-protein and the mutated sequence of Bacillus lentus DSM 5483 alkaline protease. The variant B. lentus alkaline protease M131 has the following mutations, compared to the native sequence: S3T, V41, A188P, V193M, V199I.

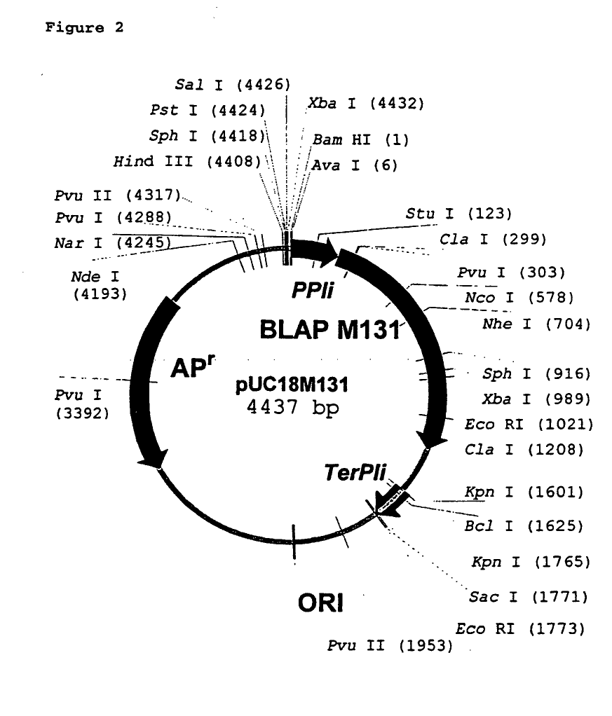

For mutagenesis, the entire expression cassette was excised by means of restriction enzymes Bam HI and Sac I and cloned into the pUC18 vector (Amersham Pharmacia Biotech, Freiburg, Germany) which had likewise been cut with Bam HI and Sac I. The pUC18M131 vector thus obtained was then used to carry out the following mutagenesis steps. FIG. 2 depicts the pUC18M131 vector. The DNA fragment containing the expression cassette for B. lentus alkaline protease M131 is documented in SEQ ID NO. 1; SEQ ID NO. 2 depicts the amino acid sequence derived therefrom. The Bam HI-SacI fragment depicted in SEQ ID NO. 1 extends over positions 1 to 1771 in the pUC18M131 vector depicted in FIG. 2; the remaining vector regions are identical to those of the starting plasmid pUC18.

Mutagenesis

First, the original sequence of Bacillus lentus DSM 5483 alkaline protease at positions 188 and 193 was restored using the QuikChange® method from Stratagene (La Jolla, Calif., USA) according to the manufacturer's instructions. According to this system, a mutated plasmid was generated in a polymerase reaction using two complementary primers containing the mutation in each case. After digesting the starting plasmid by means of DpnI, the reaction mixture was transformed into E. coli XL-1 blue. The clones obtained can, where appropriate, be readily identified by means of a restriction cleavage site introduced via the mutation, with checking by DNA sequencing according to the chain termination method with the aid of a conventional kit being possible in each case.

The triplet coding for the amino acid in position 188, CCA (proline), was converted to GCC (alanine) by using the two primers 5′-TCA CAG TAT GGC GCC GGG CTT GAC ATT-3′ and 5-AAT GTC AAG CCC GGC GCC ATA CTG TGA-3′, which contain, directly adjacent to the mutation, an Nar I restriction cleavage site which does not alter the amino acid sequence.

The triplet coding for the amino acid at position 193, ATG (methionine), was converted to ATT (isoleucine) by using the two primers 5′-GGG CTT GAC ATT GTG GCA CCC GGG GTA AAC-3′ and 5′-GTT TAC CCC GGG TGC CAC AAT GTC AAG CCC-3′ which contain, directly adjacent to the mutation, an Xma CI restriction site which does not alter the amino acid sequence.

A clone containing the doubly mutated plasmid then provided the template for subsequent mutation of the triplet at position 211, TTA (leucine) to GGA (glycine), for which the two complementary primers with the sequences 5′-ACG TAT GCT AGC GGA AAC GGT ACA TCG-3′ and 5′-CGA TGT ACC GTT TCC GCT AGC ATA CGT-3′ were used. Said sequences contain, immediately adjacent to the site of mutation, an Nhe I restriction site which does not alter the amino acid sequence. The clones obtained which produce the expected fragments using Nhe I were then checked by DNA sequencing.

The DNA sequence of the BLAP-S3T, V4I, V199I, L211G mutant gene coding for the complete protease is indicated in the sequence listing under SEQ ID NO. 3. The amino acid sequence indicated in the sequence listing under SEQ ID NO. 4 can be derived therefrom. Due to the positions deviating from the wild-type enzyme of B. lentus DSM 5483, this B. lentus alkaline protease variant is referred to as B. lentus alkaline protease S3T/V4I/V199I/L211G.

Expression of the Mutant and Protease Preparation

The expression cassette containing the mutated sequence was cloned back as Bam HI-Sac I fragment into the pCB56M131 vector, replacing the fragment depicted in SEQ ID NO. 1, and transformed into Bacillus subtilis DB104. The Bacillus subtilis DB 104 strain has the genotype his, nprR2, nprE18, aprA3 (Kawamura, F. and Doi, R. H. (1984), J. Bacteriol., volume 160, pages 442-444). The DNA was transformed into Bacillus according to the variant described in WO 91/02792 of the protoplast method originally developed by Chang and Cohen (1979; Molec. Gen. Genet., volume 168, pages 111-115).

Protease-positive clones obtained thereby were, after checking, incubated in 500 ml of MLBSP medium (10 g/l casitone; 20 g/l tryptone, 10 g/l yeast extract, all from Becton Dickinson, Cockeysville; 5 g/l NaCl; 27 g/l sodium succinate; 100 mg/l MgSO₄*7 H₂O; 75 mg/l CaCl₂*2 H₂O; 0.5 μM MnCl₂; 0.5 μM FeSO₄; 2% (w/v) glucose; 50 mM PIPES buffer (from a 1 M stock solution, pH 7.2); 75 mM KPO₄ (from a 1.5 M stock solution, pH 7.0); pH=7.0, adjusted with KOH—and 10 μg/ml tetracycline) in 2 000-ml shaker flasks at 37° C. and 200 revolutions per minute for 72 h. The supernatant obtained, after removing the cells by centrifugation, was used for the experiments below, after determining the protease activity (according to the methods described in Tenside, volume 7 (1970), pp. 125-132).

### Example 2

Textiles which had been soiled in a standardized manner and obtained from the Eidgenössische Material-Prüfungsund-Versuchsanstalt, St. Gallen, Switzerland (EMPA) or the Wäschereiforschungsanstalt, Krefeld, Germany, were used for the following two examples. The following stains/textiles were used in example 2: A (blood/milk/soot on cotton), B (blood/milk/ink on cotton), C (blood/milk/ink on a polyester-cotton blend) and D (egg/soot on cotton).

This test material was used to test the washing performances of various detergent formulations, using a launderometer. For this purpose, the liquor ratio was set in each case to 1:12, and washing was carried out at a temperature of 40° C. for 30 min. The dosage was 5.88 g of the particular detergent per 1 of wash liquor. The water hardness was 160 German hardness.

The control detergent used was a basic detergent formulation of the following composition (all values in percent by weight): 4% linear alkyl benzenesulfonate (sodium salt), 4% C₁₂-C₁₈-fatty alcohol sulfate (sodium salt), 5.5% C₁₂-C₁₈-fatty alcohol with 7 EO, 1% sodium soap, 11% sodium carbonate, 2.5% amorphous sodium disilicate, 20% sodium perborate tetrahydrate, 5.5% TAED, 25% zeolite A, 4.5% polycarboxylate, 0.5% phosphonate, 2.5% foam inhibitor granules, 5% sodium sulfate, rest: water, optical brighteners, salts. Said formulation was admixed for the various series of experiments with the following proteases in such a way that in each case a final concentration of 2.250 PE of proteolytic activity per 1 wash liquor was obtained: B. lentus alkaline protease F49 (WO 95/23221; manufacturer: Biozym, Kundl, Austria), Savinase® (Novozymes A/S, Bagsvaerd, Denmark) and the protease of the invention, B. lentus alkaline protease S3T/V4I/V199I/L211G.

After washing, the degree of whiteness of the washed textiles was measured in comparison to that of barium sulfate, which had been normalized to 100%. The measurement was carried out in a Datacolor SF500-2 spectrometer at 460 nm (UV blocking filter 3), 30 mm diaphragm, without gloss, D65 illuminant, 10°, d/8°. Table 3 below summarizes the results obtained as percent reflectance, i.e. as percentages in comparison with barium sulfate together with the respective starting values. The averages of in each case 4 measurements are listed. They allow an immediate conclusion to be drawn about the contribution of the enzyme present on the washing performance of the agent used.

TABLE 3

| Basic detergent with | A | B | C | D |
|---|---|---|---|---|
| starting value | 22.9 | 13.0 | 11.3 | 26.4 |
| Control without protease | 34.1 | 18.5 | 15.1 | 42.4 |
| B. lentus alkaline protease | 47.5 | 37.4 | 49.5 | 72.8 |
| S3T/V4I/V199I/L211G | | | | |
| B. lentus alkaline protease | 40.1 | 28.6 | 26.8 | 71.3 |
| F49 | | | | |
| Savinase ® | 43.0 | 30.5 | 29.5 | 48.6 |
| standard deviation | 0.7 | 0.7 | 1.2 | 0.9 |

The data show that the protease of the invention exhibits distinctly higher contributions to the washing performances of the particular agents on all stains than the conventional proteases B. lentus alkaline protease F49 and Savinase®.

### Example 3

In addition to the stains/textiles indicated in example 2, the sample E (blood on cotton) was used here. The test textiles were studied in the same way as in example 2 and with appropriate washing solutions in a launderometer. The only difference compared to example 2 was the fact that washing was now carried out at a temperature of 60° C. Likewise, the series of experiments were evaluated as described in the previous example; table 4 below shows the results.

TABLE 4

| Basic detergent with | A | B | C | D | E |
|---|---|---|---|---|---|
| starting value | 23.1 | 13.0 | 11.0 | 26.6 | 14.9 |
| Control without protease | 34.2 | 18.6 | 15.3 | 44.3 | 52.8 |
| B. lentus alkaline | 46.2 | 46.2 | 60.3 | 72.5 | 64.0 |
| protease | | | | | |
| S3T/V4I/V199I/L211G | | | | | |
| B. lentus alkaline | 30.7 | 30.7 | 32.2 | 72.4 | 55.3 |
| protease F49 | | | | | |
| Savinase ® | 35.9 | 35.9 | 36.4 | 56.5 | 54.0 |
| standard deviation | 0.9 | 0.9 | 1.8 | 0.8 | 1.3 |

As this result shows, the alkaline protease S3T/V4I/V199I/L211G of the invention is also at the washing temperature of 60° C. superior or, within the margin of error, at least equal to the other proteases established for detergents, B. lentus alkaline protease F49 and Savinase®.

### Example 4

Vessels with hard, smooth surfaces were contacted in a standardized way with (A) soft-boiled egg, (B) egg/milk, (C) starch mix and (D) ground meat and washed at 45° C. using the normal program of a domestic dishwasher type Miele® G 676. 20 g of dishwashing agent were used per dishwashing run; the water hardness was 16° German hardness.

The dishwashing agent used had the following basic formulation (all values in each case in percent by weight): 55% sodium tripolyphosphate (calculated as anhydrous), 4% amorphous sodium disilicate (calculated as anhydrous), 22% sodium carbonate, 9% sodium perborate, 2% TAED, 2% nonionic surfactant, rest: water, dyes, perfume. This basic formulation was admixed for the various experiments, with identical activities, with the various proteases, B. lentus alkaline protease F49, Savinase® and the protease variant of the invention, B. lentus-alkaline protease S3T/V4I/V199I/L211G, in such a way that in each case an activity of 10 000 PE per dishwashing run was obtained. This corresponded in each case to approx. 0.1 mg of protease protein per g of cleaning agent concentrate.

After washing, the removal of stains A to C was determined gravimetrically in percent. For this purpose, the difference between the weight of the soiled and then rinsed vessel and the starting weight of said vessel was related to the weight difference of the unwashed vessel to the starting weight. This relation can be regarded as percent removal. After washing, stain D was visually evaluated according to a scale from 0 (=unchanged, i.e. very heavily soiled) to 10 (=no soiling whatsoever detectable). The results obtained are summarized in table 5 below which lists the averages of in each case 9 measurements. They allow an immediate conclusion to be drawn about the contribution of the enzyme present to the washing performance of the agent used.

TABLE 5

| | A | B | C | D |
|---|---|---|---|---|
| Basic detergent with | % removal | % removal | % removal | Score |
| B. lentus alkaline | 25.2 | 27.3 | 69.3 | 9.8 |
| protease | | | | |
| S3T/V4I/V199I/L211G | | | | |
| B. lentus alkaline | 26.2 | 22.4 | 65.2 | 7.6 |
| protease F49 | | | | |
| Savinase ® | 12.5 | 12.0 | 63.3 | 8.4 |

These results show that the contribution of the B. lentus alkaline protease S3T/V4I/V199I/L211G of the invention to the cleaning performance of machine dishwashing agents is superior, but at least equal, to that of the other proteases tested; and this already at a comparatively low activity used.

### Example 5

As in the previous example, vessels were contacted with the same stains according to a standard and washed in the same way with the in each case same cleaning agent formulations. The only difference was the fact that in each case 20 000 PE of the particular proteases were used. This corresponded in each case to approx. 0.2 mg of protease in the cleaning agent concentrate. The results of the measurements, which were obtained in the same way as in example 4, are summarized in table 6 below.

TABLE 6

| | A | B | D |
|---|---|---|---|
| Basic detergent with | % removal | % removal | Score |
| B. lentus alkaline | 35.4 | 37.6 | 9.4 |
| protease | | | |
| S3T/V4I/V199I/L211G | | | |
| B. lentus alkaline | 33.2 | 32.7 | 9.1 |
| protease F49 | | | |
| Savinase ® | 12.4 | 14.0 | 8.7 |

With higher protease activities used, too, the higher contribution of the protease of the invention to the overall cleaning performance of the particular agent compared to the proteases established for machine dishwashing agents, B. lentus alkaline protease F49 and Savinase®, is evident.

Description of the figures

| | |
|---|---|
| FIG. 1: | Amino acid sequence alignment of |
| | the B. lentus alkaline protease |
| | variant of the invention with the |
| | most important known subtilisins, |
| | in each case in the mature, i.e. |
| | processed, form, in which |
| | alignment: |
| Inventive variant: | Inventive B. lentus alkaline |
| | protease variant |
| | S3T/V4I/V199I/L211G; |
| Subtilisin 309 | Bacillus lentus subtilisin |
| | according to WO 89/06279; |
| Subtilisin PB92 | Bacillus nov. spec. 92 subtilisin |
| | according to EP 283075; |
| Subtilisin Carlsberg | Bacillus licheniformis subtilisin |
| (1968), | according to E. L. Smith et al., |
| | J. Biol. Chem., Volume 243, pp. |
| | 2184-2191; |
| Subtilisin BPN' | Bacillus amyloliquefaciens |
| | subtilisin according to J. A. Wells |
| | et al. (1983), Nucleic |
| | Acids Research, Volume 11, pp. |
| | 7911-7925; |
| Consensus | Positions corresponding in the |
| | majority of the sequences |
| | indicated. |
| FIG. 2: | Mutagenesis vector pUC18M131. |
| | The Bam HI-Sac I fragment |
| | depicted in SEQ ID NO. 1 extends |
| | therein over positions 1 to 1771; |
| | the remaining vector regions are |
| | identical to those of the |
| | starting plasmid pUC18 (Amersham |
| | Pharmacia Biotech, Freiburg, |
| | Germany). |

1-35. (Canceled)

36. An alkaline protease of the subtilisin type comprising isoleucine at position 199, glycine at position 211, and at least one modification that contributes to stabilization; wherein each position corresponds to a position of the amino acid sequence of Bacillus lentus DSM 5483 subtilisin.

37. An alkaline protease of the subtilisin type comprising isoleucine at position 199, glycine at position 211, and at least one of: threonine at position 3 or isoleucine at position 4; wherein each position corresponds to a position of the amino acid sequence of Bacillus lentus DSM 5483 subtilisin.

38. An alkaline protease of the subtilisin type comprising isoleucine at position 199, glycine at position 211, threonine at position 3 and isoleucine at position 4; wherein each position corresponds to a position of the amino acid sequence of Bacillus lentus DSM 5483 subtilisin.

39. The alkaline protease of claim 36, wherein the subtilisin is derived from a Bacillus.

40. The alkaline protease of claim 39, wherein the Bacillus is Bacillus lentus.

41. The alkaline protease of claim 39, wherein the Bacillus is Bacillus lentus DSM 5483.

42. The alkaline protease of claim 41 comprising the following substitutions: S3T, V4I, V199I, and L211G.

43. A polypeptide comprising the amino acid sequence of SEQ ID NO:4 or a fragment of the amino acid sequence of SEQ ID NO:4.

44. A protein derived from the alkaline protease of claim 36 by at least one of: fragmentation mutagenesis, deletion mutagenesis, insertion mutagenesis, substitution mutagenesis or fusion of at least one part to at least one other protein.

45. The protein of claim 44 wherein the protein is additionally derivatized.

46. The protein of claim 44 wherein the protein has proteolytic activity.

47. The protein of claim 46 wherein the protein has increased proteolytic activity compared to the starting alkaline protease.

48. The protein of claim 44 wherein the protein has enhanced performance compared to the starting alkaline protease.

49. The protein of claim 44 wherein the protein is additionally stabilized.

50. An isolated nucleic acid molecule comprising a nucleotide sequence coding for the protein of claim 36.

51. An isolated nucleic acid molecule comprising the nucleotide sequence of SEQ ID NO:3 or a fragment of the nucleotide sequence of SEQ ID NO:3.

52. A vector comprising the nucleic acid molecule of claim 51.

53. The vector of claim 52 wherein the vector is a cloning vector.

54. The vector of claim 52 wherein the vector is an expression vector.

55. A cell comprising the vector of claim 52.

56. A host cell capable of expressing the alkaline protease of claim 36.

57. The host cell of claim 56 wherein the cell is a bacterium capable of secreting the alkaline protease.

58. The host cell of claim 56 wherein the bacterium is of the genus Bacillus.

59. The host cell of claim 56 wherein the bacterium is Bacillus lentus, Bacillus licheniformis, Bacillus amyloliquefaciens, Bacillus subtilis, or Bacillus alcalophilus.

60. The host cell of claim 56 wherein the cell is a eukaryotic cell.

61. The host cell of claim 56 wherein the cell is capable of modifying postranslationally the alkaline protease expressed.

62. A method for preparing an alkaline protease comprising culturing the cell of claim 56 under conditions conducive to the expression of the alkaline protease.

63. A composition comprising the alkaline protease of claim 36 and a detergent or cleaning agent.

64. The composition of claim 63 wherein the alkaline protease is present in an amount of from about 2 μg to about 20 mg per g of the composition.

65. The composition of claim 63 further comprising one or more of: additional proteases, amylases, cellulases, hemicellulases or lipases.

66. A composition for the treatment of textiles or textile raw materials comprising the alkaline protease of claim 36.

67. A method for cleaning textiles or surfaces comprising the step of activating the alkaline protease of claim 36.

68. The method of claim 67 wherein the alkaline protease is activated in an amount of from about 40 μg to about 4 g per application.

69. The method of claim 67 wherein the alkaline protease is activated in an amount of from about 400 μg to about 400 mg per application.

70. A method for the treatment of textiles or textile raw materials comprising activating the alkaline protease of claim 36.

71. The method of claim 70 wherein the textile or textile raw material being treated comprises at least one natural component.

72. The method of claim 71 wherein the natural component comprises at least one of wool or silk.

73. A method comprising activating or deactivating at least one detergent or cleaning agent ingredient, wherein the ingredient is activated or deactivated by the alkaline protease of claim 36.

74. A method comprising synthesizing or biochemically analyzing a compound using the alkaline protease of claim 36.

75. A method comprising at least one of preparing, purifying or synthesizing a biological substance using the alkaline protease of claim 36.

76. A method for the treatment of raw materials or intermediates in the manufacture of textiles comprising the step of removing a protective layer on a fabric, the step comprising contacting the layer with the alkaline protease of claim 36.

77. A method for the treatment of photographic films comprising the step of removing a protective layer on a film, the step comprising contacting the layer with the alkaline protease of claim 36.

78. A method for preparing food or animal feed comprising treating at least one of food, animal feed, or a starting substance of food or animal feed with the alkaline protease of claim 36.

79. A cosmetic composition comprising the alkaline protease of claim 36 and a suitable carrier.
